(19) United States
(12) Patent Application Publication
(10) Pub. No.: US 2023/0212599 A1
(43) Pub. Date: Jul. 6, 2023

(54) COMPOSITIONS AND METHODS FOR IMPROVED AGRONOMIC PERFORMANCE IN PLANTS

(71) Applicant: PIONEER HI-BRED INTERNATIONAL, INC., JOHNSTON, IA (US)

(72) Inventor: CARL SIMMONS, DES MOINES, IA (US)

(73) Assignee: PIONEER HI-BRED INTERNATIONAL, INC., JOHNSTON, IA (US)

(21) Appl. No.: 17/647,183

(22) Filed: Jan. 6, 2022

(51) Int. Cl. C12N 15/82

(52) U.S. Cl. CPC C12N 15/8261; C12N 15/8207; C12N 15/8205

(57) ABSTRACT

Provided are compositions comprising polynucleotides and polypeptides for the improvement of agronomic traits in plants, in particular maize plants. Also provided are recombinant DNA constructs, plants, plant cells, seed, grain comprising the polynucleotides and/or polypeptides. Additionally, various methods of employing the polynucleotides and genetic modifications in plants, such as methods for increasing yield of a plant are also provided herein.

### REFERENCE TO SEQUENCE LISTING SUBMITTED ELECTRONICALLY

The official copy of the sequence listing is submitted electronically via EFS-Web as an ASCII formatted sequence listing with a file named 8537-US-PSP_SequenceListing_ST25.txt created on 13 Nov. 2020 and having a size of 1357 kilobytes and is filed concurrently with the specification. The sequence listing comprised in this ASCII formatted document is part of the specification and is herein incorporated by reference in its entirety.

### FIELD

This disclosure relates to compositions and methods for improving agronomic traits, such as yield, in plants.

### BACKGROUND

Crop improvement has long created foundational wealth and supported societal development, human health and well-being, as well as adaptability to environmental change. Maize agronomic trait improvements have occurred for thousands of years, first supporting cultures in the Americas, and now world-wide. Crop improvement integrates diverse technologies including genetic solutions that range from traditional breeding to more recent biotechnology. Global demand and consumption of agricultural crops is increasing at a rapid pace. Accordingly, there is a need to develop new compositions and methods to increase yield in plants.

Despite advances in biotechnology, some key challenges in this type of research for product development which are commonly underappreciated. These challenges may help one better understand why despite hundreds of published reports of biotech genes improving model or crop plant productivity, and likely thousands more unpublished results, nonetheless apart from the gene performances and scientific achievements involved, very few have been yet commercialized. Importantly, despite these challenges, the efforts and results disclosed herein affirm that biotechnology can identity diverse individual genes that improve plant complex traits and yield, for example in elite germplasm, under commercially relevant field conditions.

### SUMMARY

Methods and compositions are provided for the improvement of crop plant characteristics, including those related to yield, agronomic potential, and agronomic performance.

### BRIEF DESCRIPTION OF THE SEQUENCE LISTING

The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.
SEQID NO:1 is the Secale cereale DNA sequence of RYE CBF 31.
SEQID NO:2 is the Hevea braziliensis DNA sequence of HB-PT.
SEQID NO:3 is the Hevea braziliensis DNA sequence of HB-PT.
SEQID NO:4 is the Zea mays DNA sequence of ZM-GA20OX1.
SEQID NO:5 is the Zea mays DNA sequence of ZM-MYB3.
SEQID NO:6 is the Zea mays DNA sequence of ZM-MYB3R-1.
SEQID NO:7 is the Zea mays DNA sequence of ZM-MS44.
SEQID NO:8 is the Zea mays DNA sequence of MS45.
SEQID NO:9 is the Zea mays DNA sequence of ZM-NAGS1.
SEQID NO:10 is the Arabidopsis thaliana DNA sequence of AT-FAD2.
SEQID NO:11 is the Arabidopsis thaliana DNA sequence of AT-PP2C.
SEQID NO:12 is the Arabidopsis thaliana DNA sequence of AT-DTP33.
SEQID NO:13 is the Arabidopsis thaliana DNA sequence of AT-BTIP.
SEQID NO:14 is the Arabidopsis thaliana DNA sequence of AT-NUCPU62.
SEQID NO:15 is the Arabidopsis thaliana DNA sequence of AT-IBO.
SEQID NO:16 is the Arabidopsis thaliana DNA sequence of AT-SIPR.
SEQID NO:17 is the Arabidopsis thaliana DNA sequence of AT-GSTU13.
SEQID NO:18 is the Arabidopsis thaliana DNA sequence of AT-CCO.
SEQID NO:19 is the Arabidopsis thaliana DNA sequence of AT-ESP.
SEQID NO:20 is the Arabidopsis thaliana DNA sequence of AT-SNF2.
SEQID NO:21 is the Arabidopsis thaliana DNA sequence of AT-TMK1.
SEQID NO:22 is the Arabidopsis thaliana DNA sequence of AT-NUCPU3.
SEQID NO:23 is the Arabidopsis thaliana DNA sequence of AT-DTP6.
SEQID NO:24 is the Arabidopsis thaliana DNA sequence of AT-RPK1.
SEQID NO:25 is the Arabidopsis thaliana DNA sequence of AT-NUCPU6.
SEQID NO:26 is the Arabidopsis thaliana DNA sequence of AT-40S RP-SA.
SEQID NO:27 is the Arabidopsis thaliana DNA sequence of AT-ZFP6.
SEQID NO:28 is the Arabidopsis thaliana DNA sequence of AT-C3HC4.
SEQID NO:29 is the Arabidopsis thaliana DNA sequence of AT-NUCPU7.
SEQID NO:30 is the Arabidopsis thaliana DNA sequence of AT-NDH1.
SEQID NO:31 is the Arabidopsis thaliana DNA sequence of AT-FBOX7.
SEQID NO:32 is the Arabidopsis thaliana DNA sequence of AT-NUCPU29.
SEQID NO:33 is the Arabidopsis thaliana DNA sequence of AT-NUCPU63.
SEQID NO:34 is the Arabidopsis thaliana DNA sequence of AT-ARL3.
SEQID NO:35 is the Arabidopsis thaliana DNA sequence of AT-HAD1.
SEQID NO:36 is the Arabidopsis thaliana DNA sequence of AT-TRAF.
SEQID NO:37 is the Arabidopsis thaliana DNA sequence of AT-HYDROLASE2.
SEQID NO:38 is the Arabidopsis thaliana DNA sequence of AT-DTP25.
SEQID NO:39 is the Arabidopsis thaliana DNA sequence of AT-EXST.
SEQID NO:40 is the Arabidopsis thaliana DNA sequence of AT-NUCPU45.
SEQID NO:41 is the Arabidopsis thaliana DNA sequence of AT-RBP2.
SEQID NO:42 is the Arabidopsis thaliana DNA sequence of AT-ABCG21.
SEQID NO:43 is the Arabidopsis thaliana DNA sequence of AT-TYRPPASE.
SEQID NO:44 is the Arabidopsis thaliana DNA sequence of AT-NUCPU27.
SEQID NO:45 is the Arabidopsis thaliana DNA sequence of AT-DTP5.
SEQID NO:46 is the Arabidopsis thaliana DNA sequence of AT-ETA3.
SEQID NO:47 is the Arabidopsis thaliana DNA sequence of AT-DTP7.
SEQID NO:48 is the Arabidopsis thaliana DNA sequence of AT-NUCPU80.
SEQID NO:49 is the Arabidopsis thaliana DNA sequence of AT-NUCPU81.
SEQID NO:50 is the Arabidopsis thaliana DNA sequence of AT-NUCPU34.
SEQID NO:51 is the Arabidopsis thaliana DNA sequence of AT-BHLH1.
SEQID NO:52 is the Arabidopsis thaliana DNA sequence of AT-MLP.
SEQID NO:53 is the Arabidopsis thaliana DNA sequence of AT-NDK4.
SEQID NO:54 is the Arabidopsis thaliana DNA sequence of AT-ABI1.
SEQID NO:55 is the Arabidopsis thaliana DNA sequence of AT-DTP32.
SEQID NO:56 is the Arabidopsis thaliana DNA sequence of AT-RD26-2.
SEQID NO:57 is the Arabidopsis thaliana DNA sequence of AT-RHP6.
SEQID NO:58 is the Arabidopsis thaliana DNA sequence of AT-DTP1.
SEQID NO:59 is the Arabidopsis thaliana DNA sequence of AT-PPLDD.
SEQID NO:60 is the Arabidopsis thaliana DNA sequence of AT-NUCPU74.
SEQID NO:61 is the Arabidopsis thaliana DNA sequence of AT-MYB.
SEQID NO:62 is the Arabidopsis thaliana DNA sequence of AT-PAPR.
SEQID NO:63 is the Arabidopsis thaliana DNA sequence of AT-AT5G10000/FERREDOXIN.
SEQID NO:64 is the Arabidopsis thaliana DNA sequence of AT-PEPA.
SEQID NO:65 is the Arabidopsis thaliana DNA sequence of AT-NUCPU59.
SEQID NO:66 is the Arabidopsis thaliana DNA sequence of AT-RECPU3.
SEQID NO:67 is the Arabidopsis thaliana DNA sequence of AT-PP2B12.
SEQID NO:68 is the Arabidopsis thaliana DNA sequence of AT-DTP2.
SEQID NO:69 is the Arabidopsis thaliana DNA sequence of AT-SK11.
SEQID NO:70 is the Arabidopsis thaliana DNA sequence of AT-FBOX8.
SEQID NO:71 is the Arabidopsis thaliana DNA sequence of AT-UPM1.
SEQID NO:72 is the Arabidopsis thaliana DNA sequence of AT-C3HC4 ZF2.
SEQID NO:73 is the Arabidopsis thaliana DNA sequence of AT-C3HC4 ZF2.
SEQID NO:74 is the Arabidopsis thaliana DNA sequence of AT-PBP.
SEQID NO:75 is the Arabidopsis thaliana DNA sequence of AT-C2DP1.
SEQID NO:76 is the Arabidopsis thaliana DNA sequence of AT-NUCPU1.
SEQID NO:77 is the Arabidopsis thaliana DNA sequence of AT-NUCPU1B.
SEQID NO:78 is the Arabidopsis thaliana DNA sequence of AT-LPK.
SEQID NO:79 is the Arabidopsis thaliana DNA sequence of AT-CXE20.
SEQID NO:80 is the Arabidopsis thaliana DNA sequence of AT-DTP4 (=AT-CXE20).
SEQID NO:81 is the Zea mays DNA sequence of ZM-TSCP534058.
SEQID NO:82 is the Zea mays DNA sequence of ZM-CTATP.
SEQID NO:83 is the Zea mays DNA sequence of ZM-HARDY.
SEQID NO:84 is the Zea mays DNA sequence of ZM-SIG2B.
SEQID NO:85 is the Zea mays DNA sequence of ZM-SRTF18.
SEQID NO:86 is the Zea mays DNA sequence of ZM-EIL6.
SEQID NO:87 is the Zea mays DNA sequence of ZM-UCP1.
SEQID NO:88 is the Zea mays DNA sequence of ZM-ALF1.
SEQID NO:89 is the Zea mays DNA sequence of ZM-NUCPU16.
SEQID NO:90 is the Zea mays DNA sequence of ZM-EER5.
SEQID NO:91 is the Zea mays DNA sequence of ZM-TMM.
SEQID NO:92 is the Zea mays DNA sequence of ZM-LRR-RK-P.
SEQID NO:93 is the Zea mays DNA sequence of VAC PPASE.
SEQID NO:94 is the Zea mays DNA sequence of ZM-ARP7.
SEQID NO:95 is the Zea mays DNA sequence of ZM-HP1.
SEQID NO:96 is the Zea mays DNA sequence of ZM-XERICO1.
SEQID NO:97 is the Zea mays DNA sequence of BETAEXPANSIN.
SEQID NO:98 is the Zea mays DNA sequence of ZM-MMS21.
SEQID NO:99 is the Zea mays DNA sequence of ZM-GA2 OXIDASE-LIKE.
SEQID NO:100 is the Zea mays DNA sequence of ZM-GA2 OXIDASE-LIKE PRO.
SEQID NO:101 is the Zea mays DNA sequence of ZM-XTH3.
SEQID NO:102 is the Zea mays DNA sequence of ZM-LOX6.
SEQID NO:103 is the Zea mays DNA sequence of ZM-NAP2.
SEQID NO:104 is the Zea mays DNA sequence of ZM-DOF11.
SEQID NO:105 is the Zea mays DNA sequence of ZM-TF-Q5SMY2.
SEQID NO:106 is the Zea mays DNA sequence of ZM-GS1-5.
SEQID NO:107 is the Zea mays DNA sequence of ZM-EX1.
SEQID NO:108 is the Zea mays DNA sequence of ZM-TOR.
SEQID NO:109 is the Zea mays DNA sequence of ZM-GS1-1.
SEQID NO:110 is the Zea mays DNA sequence of ZM-MACS6B.
SEQID NO:111 is the Zea mays DNA sequence of ZM-GCN2.
SEQID NO:112 is the Zea mays DNA sequence of ZM-ACO2.
SEQID NO:113 is the Zea mays DNA sequence of ZM-ASCORBATE PEROXIDASE.
SEQID NO:114 is the Zea mays DNA sequence of ZM-ACS6.
SEQID NO:115 is the Zea mays DNA sequence of ZM-MACS6A.
SEQID NO:116 is the Zea mays DNA sequence of ZM-PT5.
SEQID NO:117 is the Zea mays DNA sequence of ZM-T1N6.6.
SEQID NO:118 is the Zea mays DNA sequence of ZM-M47-V1.
SEQID NO:119 is the Zea mays DNA sequence of ZM-SVP3.
SEQID NO:120 is the Zea mays DNA sequence of ZM-PPDX.
SEQID NO:121 is the Zea mays DNA sequence of ZM-NRP1.
SEQID NO:122 is the Zea mays DNA sequence of ZM-RET2.
SEQID NO:123 is the Zea mays DNA sequence of ZM-ARGOS4.
SEQID NO:124 is the Zea mays DNA sequence of ZM-AOS1.
SEQID NO:125 is the Zea mays DNA sequence of ZM-GRF9.
SEQID NO:126 is the Zea mays DNA sequence of ZM-EIN2.
SEQID NO:127 is the Zea mays DNA sequence of ZM-AVP3.
SEQID NO:128 is the Zea mays DNA sequence of ZM-MADS56-V1.
SEQID NO:129 is the Zea mays DNA sequence of ZM-M-SV2A.
SEQID NO:130 is the Zea mays DNA sequence of ZM-LEA5.
SEQID NO:131 is the Zea mays DNA sequence of ZM-LBD1.
SEQID NO:132 is the Zea mays DNA sequence of ZM-C3HC4 RF1.
SEQID NO:133 is the Zea mays DNA sequence of ZM-RTA3.
SEQID NO:134 is the Zea mays DNA sequence of ZM-CYCLIN B2.2.
SEQID NO:135 is the Zea mays DNA sequence of ZM-RNAHEL.
SEQID NO:136 is the Zea mays DNA sequence of ZM-NPQ4.
SEQID NO:137 is the Zea mays DNA sequence of ZM-DP0780.
SEQID NO:138 is the Zea mays DNA sequence of ZM-ARABIDILLO-2.
SEQID NO:139 is the Zea mays DNA sequence of ZM-MADS32.
SEQID NO:140 is the Zea mays DNA sequence of ZM-ABA ACTIVATED KINASE1.
SEQID NO:141 is the Zea mays DNA sequence of ZM-MYB1.
SEQID NO:142 is the Zea mays DNA sequence of ZM-MYB2.
SEQID NO:143 is the Zea mays DNA sequence of ZM-MYB3R-2.
SEQID NO:144 is the Zea mays DNA sequence of ZM-PEPC.
SEQID NO:145 is the Zea mays DNA sequence of ZM-IPT2.
SEQID NO:146 is the Zea mays DNA sequence of ZM-CPCT(ALT).
SEQID NO:147 is the Zea mays DNA sequence of ZM-SDH3.
SEQID NO:148 is the Zea mays DNA sequence of SUCT.
SEQID NO:149 is the Zea mays DNA sequence of ZM-ARP6.
SEQID NO:150 is the Zea mays DNA sequence of ZM-PC4.
SEQID NO:151 is the Zea mays DNA sequence of ZM-CATALASE2.
SEQID NO:152 is the Zea mays DNA sequence of ZM-UBA-DELTA.
SEQID NO:153 is the Zea mays DNA sequence of ZM-AFB7.
SEQID NO:154 is the Zea mays DNA sequence of ZM-MCUPFB miRNA.
SEQID NO:155 is the Zea mays DNA sequence of ZM-DP0950.
SEQID NO:156 is the Zea mays DNA sequence of ZM-IPT1.
SEQID NO:157 is the Zea mays DNA sequence of ZM-IPT.
SEQID NO:158 is the Zea mays DNA sequence of ZM-PPC.
SEQID NO:159 is the Zea mays DNA sequence of ZM-PUB22.
SEQID NO:160 is the Zea mays DNA sequence of ZM-RBCS.
SEQID NO:161 is the Zea mays DNA sequence of ZM-SSU TP.
SEQID NO:162 is the Zea mays DNA sequence of ZM-PARP1.
SEQID NO:163 is the Zea mays DNA sequence of ZM-MBF1B.
SEQID NO:164 is the Zea mays DNA sequence of ZM-ZIM FP-LIKE.
SEQID NO:165 is the Zea mays DNA sequence of ZM-VRS1.
SEQID NO:166 is the Zea mays DNA sequence of ZM-ETA3.
SEQID NO:167 is the Zea mays DNA sequence of ZM-NRT1.1L2.
SEQID NO:168 is the Zea mays DNA sequence of ZM-CGS.
SEQID NO:169 is the Zea mays DNA sequence of ZM-GL1-V1.
SEQID NO:170 is the Zea mays DNA sequence of ZM-NAC7.
SEQID NO:171 is the Zea mays DNA sequence of ZM-ATG18.
SEQID NO:172 is the Zea mays DNA sequence of ZM-NAC5.
SEQID NO:173 is the Zea mays DNA sequence of ZM-ARDA.
SEQID NO:174 is the Zea mays DNA sequence of ZM-LEC1-LIKE.
SEQID NO:175 is the Zea mays DNA sequence of ZM-RR10.
SEQID NO:176 is the Zea mays DNA sequence of ZM-RR10 382-686.
SEQID NO:177 is the Zea mays DNA sequence of ZM-NAC010.
SEQID NO:178 is the Zea mays DNA sequence of ZM-ALDH7.
SEQID NO:179 is the Zea mays DNA sequence of ZM-MADS2.
SEQID NO:180 is the Zea mays DNA sequence of ZM-EBNA2.
SEQID NO:181 is the Zea mays DNA sequence of ZM-ARGOS3.
SEQID NO:182 is the Zea mays DNA sequence of ZM-EBF2.
SEQID NO:183 is the Zea mays DNA sequence of ZM-FBL2.
SEQID NO:184 is the Zea mays DNA sequence of ZM-PWWPDPL1.
SEQID NO:185 is the Zea mays DNA sequence of ZM-EDCP647011.
SEQID NO:186 is the Zea mays DNA sequence of ZM-GLYH.
SEQID NO:187 is the Zea mays DNA sequence of ZM-ODP2.
SEQID NO:188 is the Zea mays DNA sequence of ZM-MPG-V1.
SEQID NO:189 is the Zea mays DNA sequence of BRITTLE1.
SEQID NO:190 is the Zea mays DNA sequence of ZM-CHYR1-B.
SEQID NO:191 is the Zea mays DNA sequence of ZM-UPPS.
SEQID NO:192 is the Zea mays DNA sequence of ZM-ZMM28.
SEQID NO:193 is the Zea mays DNA sequence of ZM-SUS1.
SEQID NO:194 is the Zea mays DNA sequence of ZM-ALF2.
SEQID NO:195 is the Zea mays DNA sequence of ZM-HVA22E.
SEQID NO:196 is the Zea mays DNA sequence of ZM-SUMO E3.
SEQID NO:197 is the Zea mays DNA sequence of ZM-ERA1.
SEQID NO:198 is the Zea mays DNA sequence of ZM-FARNESYL TRANSF B.
SEQID NO:199 is the Zea mays DNA sequence of ZM-FARNESYL TRANSFERASE B.
SEQID NO:200 is the Zea mays DNA sequence of ZM-NAC87-2.
SEQID NO:201 is the Zea mays DNA sequence of ZM-COL.
SEQID NO:202 is the Zea mays DNA sequence of ZM-STPP3.
SEQID NO:203 is the Zea mays DNA sequence of ZM-STPP3-2 (PRO).
SEQID NO:204 is the Zea mays DNA sequence of ZM-ARGOS6.
SEQID NO:205 is the Zea mays DNA sequence of ZM-HMG.
SEQID NO:206 is the Zea mays DNA sequence of ZM-TREHALASE.
SEQID NO:207 is the Zea mays DNA sequence of ZM-WRKY67.
SEQID NO:208 is the Zea mays DNA sequence of ZM-NAR2.2.
SEQID NO:209 is the Zea mays DNA sequence of ZM-RT1.
SEQID NO:210 is the Zea mays DNA sequence of ZM-NAC1.
SEQID NO:211 is the Zea mays DNA sequence of ZM-CCCH.
SEQID NO:212 is the Zea mays DNA sequence of ZM-OBP3AL.
SEQID NO:213 is the Zea mays DNA sequence of ZM-ARGOS5.
SEQID NO:214 is the Zea mays DNA sequence of ZM-ARFGAP 1.
SEQID NO:215 is the Zea mays DNA sequence of ZM-DDA1.
SEQID NO:216 is the Zea mays DNA sequence of ZM-MYB.
SEQID NO:217 is the Zea mays DNA sequence of ZM-ADA2.
SEQID NO:218 is the Zea mays DNA sequence of ZM-GW3-1 (=ZM-BG1H1).
SEQID NO:219 is the Zea mays DNA sequence of ZM-ABA8OX1.
SEQID NO:220 is the Zea mays DNA sequence of ZM-PP2C.
SEQID NO:221 is the Zea mays DNA sequence of ZM-WILTY.
SEQID NO:222 is the Zea mays DNA sequence of ZM-EIL2.
SEQID NO:223 is the Zea mays DNA sequence of ZM-ARGOS8.
SEQID NO:224 is the Zea mays DNA sequence of ZM-WSPL1.
SEQID NO:225 is the Zea mays DNA sequence of ZM-DOF20.
SEQID NO:226 is the Zea mays DNA sequence of ZM-CBF1.
SEQID NO:227 is the Zea mays DNA sequence of ZM-RR2.
SEQID NO:228 is the Zea mays DNA sequence of ZM-XERICO2.
SEQID NO:229 is the Zea mays DNA sequence of ZM-TFL26.
SEQID NO:230 is the Zea mays DNA sequence of ZM-NUCPU3.
SEQID NO:231 is the Zea mays DNA sequence of ZM-ZF-Q75KW5.
SEQID NO:232 is the Zea mays DNA sequence of ZM-EFTU.
SEQID NO:233 is the Agrobacterium tumefaciens DNA sequence of BIP.
SEQID NO:234 is the Zea mays DNA sequence of ZM-PP2AC.
SEQID NO:235 is the Zea mays DNA sequence of ZM-BR1.
SEQID NO:236 is the Zea mays DNA sequence of ZM-EPF2A-MP.
SEQID NO:237 is the Zea mays DNA sequence of ZM-ARGOS.
SEQID NO:238 is the Zea mays DNA sequence of ZM-ARGOS1.
SEQID NO:239 is the Zea mays DNA sequence of ZM-ARGOS2.
SEQID NO:240 is the Zea mays DNA sequence of ZM-DET2.
SEQID NO:241 is the Zea mays DNA sequence of ZM-HP-Q94I52.
SEQID NO:242 is the Zea mays DNA sequence of ZM-EG8-LIKE.
SEQID NO:243 is the Zea mays DNA sequence of ZM-CKX2.
SEQID NO:244 is the Zea mays DNA sequence of ZM-YECPU2.
SEQID NO:245 is the Zea mays DNA sequence of ZM-ANR1.
SEQID NO:246 is the Zea mays DNA sequence of ZM-DRO1.
SEQID NO:247 is the Zea mays DNA sequence of ZM-DRO1.
SEQID NO:248 is the Zea mays DNA sequence of ZM-RAP2.7.
SEQID NO:249 is the Zea mays DNA sequence of ZM-GPCR.
SEQID NO:250 is the Zea mays DNA sequence of ZM-ERECTA.
SEQID NO:251 is the Zea mays DNA sequence of ZM-MYB1R1.
SEQID NO:252 is the Zea mays DNA sequence of ZM-GRF5.
SEQID NO:253 is the Zea mays DNA sequence of ZM-NAC21-5.
SEQID NO:254 is the Zea mays DNA sequence of ZM-GS1-3.
SEQID NO:255 is the Zea mays DNA sequence of ZM-SPS.
SEQID NO:256 is the Zea mays DNA sequence of ZM-MEZ2.
SEQID NO:257 is the Zea mays DNA sequence of ZM-PROG1 IR.
SEQID NO:258 is the Zea mays DNA sequence of ZM-ACC SYNTHASE 2.
SEQID NO:259 is the Zea mays DNA sequence of ZM-ACS7.
SEQID NO:260 is the Pichia angusta DNA sequence of YNT1 (PA) MO.
SEQID NO:261 is the Diplodia maydis DNA sequence of ACCD1 (DM).
SEQID NO:262 is the Artificial DNA sequence of ZM-TIC40 CTP1CB-SOR Fusion Zea mays Clostridium beijerinckii.
SEQID NO:263 is the Artificial DNA sequence of ZM-LHC 1-680 CTP1 CB-SOR (NO MET) Fusion Zea mays Clostridium beijerinckii.
SEQID NO:264 is the Zea mays DNA sequence of ZM-MSRB2.
SEQID NO:265 is the Zea mays DNA sequence of ZM-RR5.
SEQID NO:266 is the Zea mays DNA sequence of ZM-CB5A.
SEQID NO:267 is the Saccharomyces cerevisiae DNA sequence of SC-RER2.
SEQID NO:268 is the Zea mays DNA sequence of ZM-ASA1.
SEQID NO:269 is the Sorghum bicolor DNA sequence of SB-C4PEPC.
SEQID NO:270 is the Sorghum bicolor DNA sequence of SB-MADS28 GENOMIC.
SEQID NO:271 is the Clostridium beijerinckii DNA sequence of CB-SOR.
SEQID NO:272 is the Sorghum bicolor DNA sequence of SB-STPP3-4.
SEQID NO:273 is the Zea mays DNA sequence of ZM-LEA4-2.
SEQID NO:274 is the Zea mays DNA sequence of ZM-EPFL9.
SEQID NO:275 is the Ustilago maydis DNA sequence of UM-SUC2.
SEQID NO:276 is the Ustilago maydis DNA sequence of UM-03605.
SEQID NO:277 is the Zea mays DNA sequence of ZM-ABI5.
SEQID NO:278 is the Agrobacterium tumefaciens DNA sequence of IPT.
SEQID NO:279 is the Oryza sativa DNA sequence of OS-GST (DP0055).
SEQID NO:280 is the Oryza sativa DNA sequence of OS-NUECP8.
SEQID NO:281 is the Oryza sativa DNA sequence of OS-TIM17.
SEQID NO:282 is the Oryza sativa DNA sequence of OS-ARGOS(MOD1).
SEQID NO:283 is the Oryza sativa DNA sequence of OS-DP0691-V1.
SEQID NO:284 is the Oryza sativa DNA sequence of OS-MADS18.
SEQID NO:285 is the Oryza sativa DNA sequence of OS-NUECP2.
SEQID NO:286 is the Oryza sativa DNA sequence of OS-DP1013.
SEQID NO:287 is the Oryza sativa DNA sequence of OS-DP1002.
SEQID NO:288 is the Secale cereale PRT sequence of RYE CBF 31.
SEQID NO:289 is the Hevea braziliensis PRT sequence of HB-PT.
SEQID NO:290 is the Hevea braziliensis PRT sequence of HB-PT.
SEQID NO:291 is the Zea mays PRT sequence of ZM-GA20OX1.
SEQID NO:292 is the Zea mays PRT sequence of ZM-MYB3.
SEQID NO:293 is the Zea mays PRT sequence of ZM-MYB3R-1.
SEQID NO:294 is the Zea mays PRT sequence of ZM-NAGS1.
SEQID NO:295 is the Arabidopsis thaliana PRT sequence of AT-FAD2.
SEQID NO:296 is the Arabidopsis thaliana PRT sequence of AT-PP2C.
SEQID NO:297 is the Arabidopsis thaliana PRT sequence of AT-DTP33.
SEQID NO:298 is the Arabidopsis thaliana PRT sequence of AT-BTIP.
SEQID NO:299 is the Arabidopsis thaliana PRT sequence of AT-NUCPU62.
SEQID NO:300 is the Arabidopsis thaliana PRT sequence of AT-IBO.
SEQID NO:301 is the Arabidopsis thaliana PRT sequence of AT-SIPR.
SEQID NO:302 is the Arabidopsis thaliana PRT sequence of AT-GSTU13.
SEQID NO:303 is the Arabidopsis thaliana PRT sequence of AT-CCO.
SEQID NO:304 is the Arabidopsis thaliana PRT sequence of AT-ESP.
SEQID NO:305 is the Arabidopsis thaliana PRT sequence of AT-SNF2.
SEQID NO:306 is the Arabidopsis thaliana PRT sequence of AT-TMK1.
SEQID NO:307 is the Arabidopsis thaliana PRT sequence of AT-NUCPU3.
SEQID NO:308 is the Arabidopsis thaliana PRT sequence of AT-DTP6.
SEQID NO:309 is the Arabidopsis thaliana PRT sequence of AT-RPK1.
SEQID NO:310 is the Arabidopsis thaliana PRT sequence of AT-NUCPU6.
SEQID NO:311 is the Arabidopsis thaliana PRT sequence of AT-40S RP-SA.
SEQID NO:312 is the Arabidopsis thaliana PRT sequence of AT-ZFP6.
SEQID NO:313 is the Arabidopsis thaliana PRT sequence of AT-C3HC4.
SEQID NO:314 is the Arabidopsis thaliana PRT sequence of AT-NUCPU7.
SEQID NO:315 is the Arabidopsis thaliana PRT sequence of AT-NDH1.
SEQID NO:316 is the Arabidopsis thaliana PRT sequence of AT-FBOX7.
SEQID NO:317 is the Arabidopsis thaliana PRT sequence of AT-NUCPU29.
SEQID NO:318 is the Arabidopsis thaliana PRT sequence of AT-NUCPU63.
SEQID NO:319 is the Arabidopsis thaliana PRT sequence of AT-ARL3.
SEQID NO:320 is the Arabidopsis thaliana PRT sequence of AT-HAD1.
SEQID NO:321 is the Arabidopsis thaliana PRT sequence of AT-TRAF.
SEQID NO:322 is the Arabidopsis thaliana PRT sequence of AT-HYDROLASE2.
SEQID NO:323 is the Arabidopsis thaliana PRT sequence of AT-DTP25.
SEQID NO:324 is the Arabidopsis thaliana PRT sequence of AT-EXST.
SEQID NO:325 is the Arabidopsis thaliana PRT sequence of AT-NUCPU45.
SEQID NO:326 is the Arabidopsis thaliana PRT sequence of AT-RBP2.
SEQID NO:327 is the Arabidopsis thaliana PRT sequence of AT-ABCG21.
SEQID NO:328 is the Arabidopsis thaliana PRT sequence of AT-TYRPPASE.
SEQID NO:329 is the Arabidopsis thaliana PRT sequence of AT-NUCPU27.
SEQID NO:330 is the Arabidopsis thaliana PRT sequence of AT-DTP5.
SEQID NO:331 is the Arabidopsis thaliana PRT sequence of AT-ETA3.
SEQID NO:332 is the Arabidopsis thaliana PRT sequence of AT-DTP7.
SEQID NO:333 is the Arabidopsis thaliana PRT sequence of AT-NUCPU80.
SEQID NO:334 is the Arabidopsis thaliana PRT sequence of AT-NUCPU81.
SEQID NO:335 is the Arabidopsis thaliana PRT sequence of AT-NUCPU34.
SEQID NO:336 is the Arabidopsis thaliana PRT sequence of AT-BHLH1.
SEQID NO:337 is the Arabidopsis thaliana PRT sequence of AT-MLP.
SEQID NO:338 is the Arabidopsis thaliana PRT sequence of AT-NDK4.
SEQID NO:339 is the Arabidopsis thaliana PRT sequence of AT-ABI1.
SEQID NO:340 is the Arabidopsis thaliana PRT sequence of AT-DTP32.
SEQID NO:341 is the Arabidopsis thaliana PRT sequence of AT-RD26-2.
SEQID NO:342 is the Arabidopsis thaliana PRT sequence of AT-RHP6.
SEQID NO:343 is the Arabidopsis thaliana PRT sequence of AT-DTP1.
SEQID NO:344 is the Arabidopsis thaliana PRT sequence of AT-PPLDD.
SEQID NO:345 is the Arabidopsis thaliana PRT sequence of AT-NUCPU74.
SEQID NO:346 is the Arabidopsis thaliana PRT sequence of AT-MYB.
SEQID NO:347 is the Arabidopsis thaliana PRT sequence of AT-PAPR.
SEQID NO:348 is the Arabidopsis thaliana PRT sequence of AT-AT5G10000/FERREDOXIN.
SEQID NO:349 is the Arabidopsis thaliana PRT sequence of AT-PEPA.
SEQID NO:350 is the Arabidopsis thaliana PRT sequence of AT-NUCPU59.
SEQID NO:351 is the Arabidopsis thaliana PRT sequence of AT-RECPU3.
SEQID NO:352 is the Arabidopsis thaliana PRT sequence of AT-PP2B12.
SEQID NO:353 is the Arabidopsis thaliana PRT sequence of AT-DTP2.
SEQID NO:354 is the Arabidopsis thaliana PRT sequence of AT-SK11.
SEQID NO:355 is the Arabidopsis thaliana PRT sequence of AT-FBOX8.
SEQID NO:356 is the Arabidopsis thaliana PRT sequence of AT-UPM1.
SEQID NO:357 is the Arabidopsis thaliana PRT sequence of AT-C3HC4 ZF2.
SEQID NO:358 is the Arabidopsis thaliana PRT sequence of AT-C3HC4 ZF2.
SEQID NO:359 is the Arabidopsis thaliana PRT sequence of AT-PBP.
SEQID NO:360 is the Arabidopsis thaliana PRT sequence of AT-C2DP1.
SEQID NO:361 is the Arabidopsis thaliana PRT sequence of AT-NUCPU1.
SEQID NO:362 is the Arabidopsis thaliana PRT sequence of AT-NUCPU1B.
SEQID NO:363 is the Arabidopsis thaliana PRT sequence of AT-LPK.
SEQID NO:364 is the Arabidopsis thaliana PRT sequence of AT-CXE20.
SEQID NO:365 is the Arabidopsis thaliana PRT sequence of AT-DTP4 (=AT-CXE20).
SEQID NO:366 is the Zea mays PRT sequence of ZM-CTATP.
SEQID NO:367 is the Zea mays PRT sequence of ZM-HARDY.
SEQID NO:368 is the Zea mays PRT sequence of ZM-SIG2B.
SEQID NO:369 is the Zea mays PRT sequence of ZM-SRTF18.
SEQID NO:370 is the Zea mays PRT sequence of ZM-EIL6.
SEQID NO:371 is the Zea mays PRT sequence of ZM-UCP1.
SEQID NO:372 is the Zea mays PRT sequence of ZM-ALF1.
SEQID NO:373 is the Zea mays PRT sequence of ZM-NUCPU16.
SEQID NO:374 is the Zea mays PRT sequence of ZM-EER5.
SEQID NO:375 is the Zea mays PRT sequence of ZM-TMM.
SEQID NO:376 is the Zea mays PRT sequence of ZM-LRR-RK-P.
SEQID NO:377 is the Zea mays PRT sequence of VAC PPASE.
SEQID NO:378 is the Zea mays PRT sequence of ZM-ARP7.
SEQID NO:379 is the Zea mays PRT sequence of ZM-HP1.
SEQID NO:380 is the Zea mays PRT sequence of ZM-XERICO1.
SEQID NO:381 is the Zea mays PRT sequence of BETAEXPANSIN.
SEQID NO:382 is the Zea mays PRT sequence of ZM-MMS21.
SEQID NO:383 is the Zea mays PRT sequence of ZM-GA2 OXIDASE-LIKE.
SEQID NO:384 is the Zea mays PRT sequence of ZM-GA2 OXIDASE-LIKE PRO.
SEQID NO:385 is the Zea mays PRT sequence of ZM-XTH3.
SEQID NO:386 is the Zea mays PRT sequence of ZM-LOX6.
SEQID NO:387 is the Zea mays PRT sequence of ZM-NAP2.
SEQID NO:388 is the Zea mays PRT sequence of ZM-DOF11.
SEQID NO:389 is the Zea mays PRT sequence of ZM-TF-Q5SMY2.
SEQID NO:390 is the Zea mays PRT sequence of ZM-GS1-5.
SEQID NO:391 is the Zea mays PRT sequence of ZM-EX1.
SEQID NO:392 is the Zea mays PRT sequence of ZM-TOR.
SEQID NO:393 is the Zea mays PRT sequence of ZM-GS1-1.
SEQID NO:394 is the Zea mays PRT sequence of ZM-MACS6B.
SEQID NO:395 is the Zea mays PRT sequence of ZM-GCN2.
SEQID NO:396 is the Zea mays PRT sequence of ZM-ACO2.
SEQID NO:397 is the Zea mays PRT sequence of ZM-ASCORBATE PEROXIDASE.
SEQID NO:398 is the Zea mays PRT sequence of ZM-ACS6.
SEQID NO:399 is the Zea mays PRT sequence of ZM-MACS6A.
SEQID NO:400 is the Zea mays PRT sequence of ZM-PT5.
SEQID NO:401 is the Zea mays PRT sequence of ZM-T1N6.6.
SEQID NO:402 is the Zea mays PRT sequence of ZM-M47-V1.
SEQID NO:403 is the Zea mays PRT sequence of ZM-SVP3.
SEQID NO:404 is the Zea mays PRT sequence of ZM-PPDX.
SEQID NO:405 is the Zea mays PRT sequence of ZM-NRP1.
SEQID NO:406 is the Zea mays PRT sequence of ZM-RET2.
SEQID NO:407 is the Zea mays PRT sequence of ZM-ARGOS4.
SEQID NO:408 is the Zea mays PRT sequence of ZM-A0S1.
SEQID NO:409 is the Zea mays PRT sequence of ZM-GRF9.
SEQID NO:410 is the Zea mays PRT sequence of ZM-EIN2.
SEQID NO:411 is the Zea mays PRT sequence of ZM-AVP3.
SEQID NO:412 is the Zea mays PRT sequence of ZM-MADS56-V1.
SEQID NO:413 is the Zea mays PRT sequence of ZM-M-SV2A.
SEQID NO:414 is the Zea mays PRT sequence of ZM-LEA5.
SEQID NO:415 is the Zea mays PRT sequence of ZM-LBD1.
SEQID NO:416 is the Zea mays PRT sequence of ZM-C3HC4 RF1.
SEQID NO:417 is the Zea mays PRT sequence of ZM-RTA3.
SEQID NO:418 is the Zea mays PRT sequence of ZM-CYCLIN B2.2.
SEQID NO:419 is the Zea mays PRT sequence of ZM-RNAHEL.
SEQID NO:420 is the Zea mays PRT sequence of ZM-NPQ4.
SEQID NO:421 is the Zea mays PRT sequence of ZM-DP0780.
SEQID NO:422 is the Zea mays PRT sequence of ZM-ARABIDILLO-2.
SEQID NO:423 is the Zea mays PRT sequence of ZM-MADS32.
SEQID NO:424 is the Zea mays PRT sequence of ZM-ABA ACTIVATED KINASE1.
SEQID NO:425 is the Zea mays PRT sequence of ZM-MYB1.
SEQID NO:426 is the Zea mays PRT sequence of ZM-MYB2.
SEQID NO:427 is the Zea mays PRT sequence of ZM-MYB3R-2.
SEQID NO:428 is the Zea mays PRT sequence of ZM-PEPC.
SEQID NO:429 is the Zea mays PRT sequence of ZM-IPT2.
SEQID NO:430 is the Zea mays PRT sequence of ZM-CPCT(ALT).
SEQID NO:431 is the Zea mays PRT sequence of ZM-SDH3.
SEQID NO:432 is the Zea mays PRT sequence of SUCT.
SEQID NO:433 is the Zea mays PRT sequence of ZM-ARP6.
SEQID NO:434 is the Zea mays PRT sequence of ZM-PC4.
SEQID NO:435 is the Zea mays PRT sequence of ZM-CATALASE2.
SEQID NO:436 is the Zea mays PRT sequence of ZM-UBA-DELTA.
SEQID NO:437 is the Zea mays PRT sequence of ZM-AFB7.
SEQID NO:438 is the Zea mays PRT sequence of ZM-MCUPFB miRNA.
SEQID NO:439 is the Zea mays PRT sequence of ZM-DP0950.
SEQID NO:440 is the Zea mays PRT sequence of ZM-IPT1.
SEQID NO:441 is the Zea mays PRT sequence of ZM-IPT.
SEQID NO:442 is the Zea mays PRT sequence of ZM-PPC.
SEQID NO:443 is the Zea mays PRT sequence of ZM-PUB22.
SEQID NO:444 is the Zea mays PRT sequence of ZM-RBCS.
SEQID NO:445 is the Zea mays PRT sequence of ZM-SSU TP.
SEQID NO:446 is the Zea mays PRT sequence of ZM-PARP1.
SEQID NO:447 is the Zea mays PRT sequence of ZM-MBF1B.
SEQID NO:448 is the Zea mays PRT sequence of ZM-ZIM FP-LIKE.
SEQID NO:449 is the Zea mays PRT sequence of ZM-VRS1.
SEQID NO:450 is the Zea mays PRT sequence of ZM-ETA3.
SEQID NO:451 is the Zea mays PRT sequence of ZM-NRT1.1L2.
SEQID NO:452 is the Zea mays PRT sequence of ZM-CGS.
SEQID NO:453 is the Zea mays PRT sequence of ZM-GL1-V1.
SEQID NO:454 is the Zea mays PRT sequence of ZM-NAC7.
SEQID NO:455 is the Zea mays PRT sequence of ZM-ATG18.
SEQID NO:456 is the Zea mays PRT sequence of ZM-NAC5.
SEQID NO:457 is the Zea mays PRT sequence of ZM-ARDA.
SEQID NO:458 is the Zea mays PRT sequence of ZM-LEC1-LIKE.
SEQID NO:459 is the Zea mays PRT sequence of ZM-RR10.
SEQID NO:460 is the Zea mays PRT sequence of ZM-RR10 382-686.
SEQID NO:461 is the Zea mays PRT sequence of ZM-NAC010.
SEQID NO:462 is the Zea mays PRT sequence of ZM-ALDH7.
SEQID NO:463 is the Zea mays PRT sequence of ZM-MADS2.
SEQID NO:464 is the Zea mays PRT sequence of ZM-EBNA2.
SEQID NO:465 is the Zea mays PRT sequence of ZM-ARGOS3.
SEQID NO:466 is the Zea mays PRT sequence of ZM-EBF2.
SEQID NO:467 is the Zea mays PRT sequence of ZM-FBL2.
SEQID NO:468 is the Zea mays PRT sequence of ZM-PWWPDPL1.
SEQID NO:469 is the Zea mays PRT sequence of ZM-EDCP647011.
SEQID NO:470 is the Zea mays PRT sequence of ZM-GLYH.
SEQID NO:471 is the Zea mays PRT sequence of ZM-ODP2.
SEQID NO:472 is the Zea mays PRT sequence of ZM-MPG-V1.
SEQID NO:473 is the Zea mays PRT sequence of BRITTLE1.
SEQID NO:474 is the Zea mays PRT sequence of ZM-CHYR1-B.
SEQID NO:475 is the Zea mays PRT sequence of ZM-UPPS.
SEQID NO:476 is the Zea mays PRT sequence of ZM-ZMM28.
SEQID NO:477 is the Zea mays PRT sequence of ZM-SUS1.
SEQID NO:478 is the Zea mays PRT sequence of ZM-ALF2.
SEQID NO:479 is the Zea mays PRT sequence of ZM-HVA22E.
SEQID NO:480 is the Zea mays PRT sequence of ZM-SUMO E3.
SEQID NO:481 is the Zea mays PRT sequence of ZM-ERA1.
SEQID NO:482 is the Zea mays PRT sequence of ZM-FARNESYL TRANSF B.
SEQID NO:483 is the Zea mays PRT sequence of ZM-FARNESYL TRANSFERASE B.
SEQID NO:484 is the Zea mays PRT sequence of ZM-NAC87-2.
SEQID NO:485 is the Zea mays PRT sequence of ZM-COL.
SEQID NO:486 is the Zea mays PRT sequence of ZM-STPP3.
SEQID NO:487 is the Zea mays PRT sequence of ZM-ARGOS6.
SEQID NO:488 is the Zea mays PRT sequence of ZM-HMG.
SEQID NO:489 is the Zea mays PRT sequence of ZM-TREHALASE.
SEQID NO:490 is the Zea mays PRT sequence of ZM-WRKY67.
SEQID NO:491 is the Zea mays PRT sequence of ZM-NAR2.2.
SEQID NO:492 is the Zea mays PRT sequence of ZM-RT1.
SEQID NO:493 is the Zea mays PRT sequence of ZM-NAC1.
SEQID NO:494 is the Zea mays PRT sequence of ZM-CCCH.
SEQID NO:495 is the Zea mays PRT sequence of ZM-OBP3AL.
SEQID NO:496 is the Zea mays PRT sequence of ZM-ARGOS5.
SEQID NO:497 is the Zea mays PRT sequence of ZM-ARFGAP 1.
SEQID NO:498 is the Zea mays PRT sequence of ZM-DDA1.
SEQID NO:499 is the Zea mays PRT sequence of ZM-MYB.
SEQID NO:500 is the Zea mays PRT sequence of ZM-ADA2.
SEQID NO:501 is the Zea mays PRT sequence of ZM-GW3-1 (=ZM-BG1H1).
SEQID NO:502 is the Zea mays PRT sequence of ZM-ABA8OX1.
SEQID NO:503 is the Zea mays PRT sequence of ZM-PP2C.
SEQID NO:504 is the Zea mays PRT sequence of ZM-WILTY.
SEQID NO:505 is the Zea mays PRT sequence of ZM-EIL2.
SEQID NO:506 is the Zea mays PRT sequence of ZM-ARGOS8.
SEQID NO:507 is the Zea mays PRT sequence of ZM-WSPL1.
SEQID NO:508 is the Zea mays PRT sequence of ZM-DOF20.
SEQID NO:509 is the Zea mays PRT sequence of ZM-CBF1.
SEQID NO:510 is the Zea mays PRT sequence of ZM-RR2.
SEQID NO:511 is the Zea mays PRT sequence of ZM-XERICO2.
SEQID NO:512 is the Zea mays PRT sequence of ZM-TFL26.
SEQID NO:513 is the Zea mays PRT sequence of ZM-NUCPU3.
SEQID NO:514 is the Zea mays PRT sequence of ZM-ZF-Q75KW5.
SEQID NO:515 is the Zea mays PRT sequence of ZM-EFTU.
SEQID NO:516 is the Agrobacterium tumefaciens PRT sequence of BIP.
SEQID NO:517 is the Zea mays PRT sequence of ZM-PP2AC.
SEQID NO:518 is the Zea mays PRT sequence of ZM-BR1.
SEQID NO:519 is the Zea mays PRT sequence of ZM-EPF2A-MP.
SEQID NO:520 is the Zea mays PRT sequence of ZM-ARGOS.
SEQID NO:521 is the Zea mays PRT sequence of ZM-ARGOS1.
SEQID NO:522 is the Zea mays PRT sequence of ZM-ARGOS2.
SEQID NO:523 is the Zea mays PRT sequence of ZM-DET2.
SEQID NO:524 is the Zea mays PRT sequence of ZM-HP-Q94I52.
SEQID NO:525 is the Zea mays PRT sequence of ZM-EG8-LIKE.
SEQID NO:526 is the Zea mays PRT sequence of ZM-CKX2.
SEQID NO:527 is the Zea mays PRT sequence of ZM-YECPU2.
SEQID NO:528 is the Zea mays PRT sequence of ZM-ANR1.
SEQID NO:529 is the Zea mays PRT sequence of ZM-DRO1.
SEQID NO:530 is the Zea mays PRT sequence of ZM-DRO1.
SEQID NO:531 is the Zea mays PRT sequence of ZM-RAP2.7.
SEQID NO:532 is the Zea mays PRT sequence of ZM-GPCR.
SEQID NO:533 is the Zea mays PRT sequence of ZM-ERECTA.
SEQID NO:534 is the Zea mays PRT sequence of ZM-MYB1R1.
SEQID NO:535 is the Zea mays PRT sequence of ZM-GRF5.
SEQID NO:536 is the Zea mays PRT sequence of ZM-NAC21-5.
SEQID NO:537 is the Zea mays PRT sequence of ZM-GS1-3.
SEQID NO:538 is the Zea mays PRT sequence of ZM-SPS.
SEQID NO:539 is the Zea mays PRT sequence of ZM-MEZ2.
SEQID NO:540 is the Zea mays PRT sequence of ZM-PROG1 IR.
SEQID NO:541 is the Pichia angusta PRT sequence of YNT1 (PA) MO.
SEQID NO:542 is the Diplodia maydis PRT sequence of ACCD1 (DM).
SEQID NO:543 is the Artificial PRT sequence of ZM-TIC40 CTP1::CB-SOR Fusion
Zea mays; Clostridium beijerinckii.
SEQID NO:544 is the Artificial PRT sequence of ZM-LHC 1-680 CTP1::CB-SOR (NO MET) Fusion Zea mays; Clostridium beijerinckii.
SEQID NO:545 is the Zea mays PRT sequence of ZM-MSRB2.
SEQID NO:546 is the Zea mays PRT sequence of ZM-RR5.
SEQID NO:547 is the Zea mays PRT sequence of ZM-CB5A.
SEQID NO:548 is the Saccharomyces cerevisiae PRT sequence of SC-RER2.
SEQID NO:549 is the Zea mays PRT sequence of ZM-ASA1.
SEQID NO:550 is the Sorghum bicolor PRT sequence of SB-C4PEPC.
SEQID NO:551 is the Clostridium beijerinckii PRT sequence of CB-SOR.
SEQID NO:552 is the Sorghum bicolor PRT sequence of SB-STPP3-4.
SEQID NO:553 is the Zea mays PRT sequence of ZM-LEA4-2.
SEQID NO:554 is the Zea mays PRT sequence of ZM-EPFL9.
SEQID NO:555 is the Ustilago maydis PRT sequence of UM-SUC2.
SEQID NO:556 is the Ustilago maydis PRT sequence of UM-03605.
SEQID NO:557 is the Zea mays PRT sequence of ZM-ABI5.
SEQID NO:558 is the Agrobacterium tumefaciens PRT sequence of IPT.
SEQID NO:559 is the Oryza sativa PRT sequence of OS-GST (DP0055).
SEQID NO:560 is the Oryza sativa PRT sequence of OS-NUECP8.
SEQID NO:561 is the Oryza sativa PRT sequence of OS-TIM17.
SEQID NO:562 is the Oryza sativa PRT sequence of OS-ARGOS(MOD1).
SEQID NO:563 is the Oryza sativa PRT sequence of OS-DP0691-V1.
SEQID NO:564 is the Oryza sativa PRT sequence of OS-MADS18.
SEQID NO:565 is the Oryza sativa PRT sequence of OS-NUECP2.
SEQID NO:566 is the Oryza sativa PRT sequence of OS-DP1013.
SEQID NO:567 is the Oryza sativa PRT sequence of OS-DP1002.

### DETAILED DESCRIPTION

Terms used in the claims and specification are defined as set forth below unless otherwise specified. It must be noted that, as used in the specification and the appended claims, the singular forms “a,” “an” and “the” include plural referents unless the context clearly dictates otherwise.

The term ‘Agronomic Traits’ has varied definitions. Herein we use the term Agronomic traits chiefly to loosely refer to trait targets yield enhancement (YE), drought tolerance (DRT), and nitrogen use efficiency (NUE), all three of which have varied definitions and complex interrelationships. Occasionally it includes other component traits involving plant architecture, maturity traits, and physiological traits driving intrinsic plant performances such as photosynthetic efficiency. Collectively Agronomic Traits broadly includes many mechanisms, whether molecular or physiological, touching diverse aspects of plant biology.

The present disclosure provides a commodity plant product, as well as methods for producing a commodity plant product, that is derived from a plant of the present disclosure. As used herein, a “commodity plant product” refers to any composition or product that is comprised of material derived from a plant, seed, plant cell, or plant element of the present disclosure. Commodity plant products may be sold to consumers and can be viable or nonviable. Nonviable commodity products include but are not limited to nonviable plant elements and grains; processed seeds, seed parts, and plant elements; dehydrated plant tissue, frozen plant tissue, and processed plant tissue; seeds and plant elements processed for animal feed for terrestrial and/or aquatic animal consumption, oil, meal, flour, flakes, bran, fiber, paper, tea, coffee, silage, crushed of whole grain, and any other food for human or animal consumption such as the fruit or other edible portion of the plant; and biomasses and fuel products; and raw material in industry.

“Biomass” means the total mass or weight (fresh or dry), at a given time, of a plant tissue, plant tissues, an entire plant, or population of plants. Biomass is usually given as weight per unit area.

The term “isoline” is a comparative term, and references organisms that are genetically identical, but may differ in treatment. In one example, two genetically identical maize plant embryos may be separated into two different groups, one receiving a treatment (such as transformation with a heterologous polynucleotide, to create a genetically modified plant) and one control that does not receive such treatment. Any phenotypic differences between the two groups may thus be attributed solely to the treatment and not to any inherency of the plant's genetic makeup. Any phenotypic differences between the plants derived from (e.g., grown from or obtained from) those seeds may be attributed to the treatment, thus forming an isoline comparison.

Similarly, by the term “reference agricultural plant,” it is meant an agricultural plant of the same species, strain, or cultivar to which a transgene, treatment, formulation, composition or preparation as described herein is not administered/contacted. A reference agricultural plant, therefore, is identical to the treated plant with the exception of the presence of the transgene and can serve as a control for detecting the effects of the transgene that is conferred to the plant.

A “reference environment” refers to the environment, treatment or condition of the plant in which a measurement is made. For example, production of a compound in a plant associated with a transgene can be measured in a reference environment of drought stress, and compared with the levels of the compound in a reference agricultural plant under the same conditions of drought stress. Alternatively, the levels of a compound in plant associated with a transgene and reference agricultural plant can be measured under identical conditions of no stress.

A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, kelkis, shoot, bud. As used herein, a “plant element” is synonymous to a “portion” of a plant, and refers to any part of the plant, and can include distinct tissues and/or organs, and may be used interchangeably with the term “tissue” throughout.

Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keikis, or bud.

A “population” of plants refers to more than one plant, that are of the same taxonomic category, typically be of the same species, and will also typically share a common genetic derivation.

As used herein, the terms “water-limited condition” and “drought condition,” or “water-limited” and “drought,” may be used interchangeably. For example, a method or composition for improving a plant's ability to grow under drought conditions means the same as the ability to grow under water-limited conditions. In such cases, the plant can be further said to display improved tolerance to drought stress.

As used herein, the terms “normal watering” and “well-watered” are used interchangeably, to describe a plant grown under typical growth conditions with no water restriction.

An “increased yield” can refer to any increase in biomass or seed or fruit weight, seed size, seed number per plant, seed number per unit area, bushels per acre, tons per acre, kilo per hectare, or carbohydrate yield. Typically, the particular characteristic is designated when referring to increased yield, e.g., increased grain yield or increased seed size.

“Agronomic trait potential” is intended to mean a capability of a plant element for exhibiting a phenotype, preferably an improved agronomic trait, at some point during its life cycle, or conveying said phenotype to another plant element with which it is associated in the same plant. For example, a plant element may comprise a transgene that will provide benefit to leaf tissue of a plant from which the plant element is grown; in such case, the plant element comprising such a transgene has the agronomic trait potential for a particular phenotype (for example, increased biomass in the plant) even if the plant element itself does not display said phenotype.

“Introducing” is intended to mean presenting to the host cell, plant, plant cell or plant part the polynucleotide or polypeptide in such a manner that the sequence gains access to the interior of a cell of the plant or organism

“Heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

“Isolated” nucleic acid molecule (or DNA) is used herein to refer to a nucleic acid sequence (or DNA) that is no longer in its natural environment, for example in an in vitro or in a heterologous recombinant bacterial or plant host cell. An isolated nucleic acid molecule, or biologically active portion thereof, is substantially free of other cellular material or culture medium when produced by recombinant techniques, or substantially free of chemical precursors or other chemicals when chemically synthesized. An isolated nucleic acid is free of sequences (optimally protein encoding sequences) that naturally flank the nucleic acid (i.e., sequences located at the 5′ and 3′ ends of the nucleic acid) in the genomic DNA of the organism from which the nucleic acid is derived.

“Operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation

“Polynucleotide” is not intended to limit the present disclosure to polynucleotides comprising DNA or RNA. Such deoxyribonucleotides and ribonucleotides include both naturally occurring molecules and synthetic analogues. The polynucleotides disclosed herein also encompass all forms of sequences including, but not limited to, single-stranded forms, double-stranded forms, hairpins, stem-and-loop structures, and the like.

By “promoter” is intended a regulatory region of DNA capable of regulating the transcription of a sequence operably linked thereto. It usually comprises a TATA box capable of directing RNA polymerase II to initiate RNA synthesis at the appropriate transcription initiation site for a particular coding sequence.

“Recombinant” refers to an artificial combination of two otherwise separated segments of sequence, e.g., by chemical synthesis, or manipulation of isolated segments of nucleic acids by genetic engineering techniques.

“Percent (%) sequence identity” or “percent identity”, with respect to a reference sequence (subject), is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

“Introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. Offspring comprising the desired allele may be repeatedly backcrossed to a line having a desired genetic background and selected for the desired allele, to result in the allele becoming fixed in a selected genetic background.

“Plant” generically includes whole plants, plant organs, plant tissues, seeds, plant cells, seeds and progeny of the same. The plant is a monocot or dicot. Plant cells include, without limitation, cells from seeds, suspension cultures, embryos, meristematic regions, callus tissue, leaves, roots, shoots, gametophytes, sporophytes, pollen and microspores. A plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). It should be noted that a protoplast is not technically “intact” plant cell (as naturally found with all components), as protoplasts lack a cell wall. plant organ” refers to plant tissue or a group of tissues that constitute a morphologically and functionally distinct part of a plant. a plant element” is synonymous to a portion” of a plant, and refers to any part of the plant, and can include distinct tissues and/or organs, and may be used interchangeably with tissue” throughout. Similarly, a plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

“Subject plant” or “subject plant cell” is one in which genetic alteration, such as transformation, has been effected as to a gene of interest, or is a plant or plant cell which is descended from a plant or cell so altered and which comprises the alteration.

“Stable transformation” is a transformation in which the polynucleotide construct introduced into a plant integrates into the genome of the plant and is capable of being inherited by the progeny thereof

“Transient transformation” means that a polynucleotide is introduced into the plant and does not integrate into the genome of the plant.

“Trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance.

### Recombinant Constructs and Transformation of Cells

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, N.Y. (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

### Polynucleotides of Interest

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

General categories of polynucleotides of interest include, for example, genes of interest involved in information, such as zinc fingers, those involved in communication, such as kinases, and those involved in housekeeping, such as heat shock proteins. More specific polynucleotides of interest include, but are not limited to, genes involved in traits of agronomic interest such as but not limited to: crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to toxins such as pesticides and herbicides), genes encoding proteins conferring resistance to biotic stress (such as attacks by fungi, viruses, bacteria, insects, and nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS, also referred to as acetohydroxyacid synthase, AHAS), in particular the sulfonylurea (UK: sulphonylurea) type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077, 5,310,667, 5,866,775, 6,225,114, 6,248,876, 7,169,970, 6,867,293, and 9,187,762. The bar gene encodes resistance to the herbicide basta, the nptII gene encodes resistance to the antibiotics kanamycin and geneticin, and the ALS-gene mutants encode resistance to the herbicide chlorsulfuron.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See U.S. Pat. Nos. 5,283,184 and 5,034,323.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones (Shaner and Singh, 1997, Herbicide Activity: Toxicol Biochem Mol Biol 69-110); glyphosate resistant 5-enolpyruvylshikimate-3-phosphate (EPSPS) (Saroha et al. 1998, J. Plant Biochemistry & Biotechnology Vol 7:65-72);

Polynucleotides of interest includes genes that can be stacked or used in combination with other traits, such as but not limited to herbicide resistance or any other trait described herein. Polynucleotides of interest and/or traits can be stacked together in a complex trait locus as described in US20130263324 published 3 Oct. 2013 and in WO/2013/112686, published 1 Aug. 2013.

A polypeptide of interest includes any protein or polypeptide that is encoded by a polynucleotide of interest described herein.

Further provided are methods for identifying at least one plant cell, comprising in its genome, a polynucleotide of interest integrated at the target site. A variety of methods are available for identifying those plant cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, US20090133152 published 21 May 2009. The method also comprises recovering a plant from the plant cell comprising a polynucleotide of interest integrated into its genome. The plant may be sterile or fertile. It is recognized that any polynucleotide of interest can be provided, integrated into the plant genome at the target site, and expressed in a plant.

### Optimization of Sequences for Expression in Plants

Methods are available in the art for synthesizing plant-preferred genes. See, for example, U.S. Pat. Nos. 5,380,831, and 5,436,391, and Murray et al. (1989) Nucleic Acids Res. 17:477-498. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

### Expression Elements

Any polynucleotide encoding a Cas protein, other CRISPR system component, or other polynucleotide disclosed herein may be functionally linked to a heterologous expression element, to facilitate transcription or regulation in a host cell. Such expression elements include but are not limited to: promoter, leader, intron, and terminator. Expression elements may be “minimal”—meaning a shorter sequence derived from a native source, that still functions as an expression regulator or modifier. Alternatively, an expression element may be “optimized”—meaning that its polynucleotide sequence has been altered from its native state in order to function with a more desirable characteristic in a particular host cell (for example, but not limited to, a bacterial promoter may be “maize-optimized” to improve its expression in corn plants). Alternatively, an expression element may be “synthetic”—meaning that it is designed in silico and synthesized for use in a host cell. Synthetic expression elements may be entirely synthetic, or partially synthetic (comprising a fragment of a naturally-occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In vitro Cell Dev Biol 40:1-22; Porto et al., 2014, Molecular Biotechnology (2014), 56(1), 38-49.

Constitutive promoters include, for example, the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; ALS promoter (U.S. Pat. No. 5,659,026) and the like. Tissue-preferred promoters can be utilized to target enhanced expression within a particular plant tissue. Seed-preferred promoters include both seed-specific promoters active during seed development, as well as seed-germinating promoters active during seed germination. Chemical inducible (regulated) promoters can be used to modulate the expression of a gene in a prokaryotic and eukaryotic cell or organism through the application of an exogenous chemical regulator. The promoter may be a chemical-inducible promoter, where application of the chemical induces gene expression, or a chemical-repressible promoter, where application of the chemical represses gene expression. Pathogen inducible promoters induced following infection by a pathogen include, but are not limited to those regulating expression of PR proteins, SAR proteins, beta-1,3-glucanase, chitinase, etc. A stress-inducible promoter includes the RD29A promoter (Kasuga et al. (1999) Nature Biotechnol. 17:287-91). One of ordinary skill in the art is familiar with protocols for simulating stress conditions such as drought, osmotic stress, salt stress and temperature stress and for evaluating stress tolerance of plants that have been subjected to simulated or naturally-occurring stress conditions.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, N.Y.: Academic Press), pp. 1-82.

### Morphogenic Factors

A morphogenic factor (gene or protein)—also referred to as a “developmental gene”—may be involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof. Introduction of one or more morphogenic factors may improve the frequency or efficiency of transformation or embryogenesis.

In some aspects, the morphogenic factor is a molecule selected from one or more of the following categories: 1) cell cycle stimulatory polynucleotides including plant viral replicase genes such as RepA, cyclins, E2F, prolifera, cdc2 and cdc25; 2) developmental polynucleotides such as Lec1, Kn1 family, WUSCHEL, Zwille, BBM, Aintegumenta (ANT), FUS3, and members of the Knotted family, such as Kn1, STM, OSH1, and SbH1; 3) anti-apoptosis polynucleotides such as CED9, Bcl2, Bcl-X(L), Bcl-W, A1, McL-1, Mac1, Boo, and Bax-inhibitors; 4) hormone polynucleotides such as IPT, TZS, and CKI-1; and 5) silencing constructs targeted against cell cycle repressors, such as Rb, CK1, prohibitin, and wee1, or stimulators of apoptosis such as APAF-1, bad, bax, CED-4, and caspase-3, and repressors of plant developmental transitions, such as Pickle and WD polycomb genes including FIE and Medea. The polynucleotides can be silenced by any known method such as antisense, RNA interference, cosuppression, chimerplasty, or transposon insertion.

In some aspects, the morphogenic gene is a member of the WUS/WOX gene family (WUS1, WUS2, WUS3, WOX2A, WOX4, WOX5, or WOX9) see U.S. Pat. Nos. 7,348,468 and 7,256,322 and United States Patent Application publications 20170121722 and 20070271628.

In some embodiments, the morphogenic gene or protein is a member of the AP2/ERF family of proteins.

In some embodiments, the morphogenic factor is a babyboom (BBM) polypeptide, which is a member of the AP2 family of transcription factors.

### Cas Endonucleases and Effectors

Endonucleases are enzymes that cleave the phosphodiester bond within a polynucleotide chain, and include restriction endonucleases that cleave DNA at specific sites without damaging the bases. Examples of endonucleases include restriction endonucleases, meganucleases, TAL effector nucleases (TALENs), zinc finger nucleases, and Cas (CRISPR-associated) effector endonucleases.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases may occur as individual effectors (Class 2 CRISPR systems) or as part of larger effector complexes (Class I CRISPR systems).

Cas endonucleases that have been described include, but are not limited to, for example: Cas3 (a feature of Class 1 type I systems), Cas9 (a feature of Class 2 type II systems) and Cas12 (Cpf1) (a feature of Class 2 type V systems).

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10:957-963).

Cas endonucleases, when complexed with a cognate guide RNA, recognize, bind to, and optionally nick or cleave a target polynucleotide.

A Cas endonuclease, effector protein, or functional fragment thereof, for use in the disclosed methods, can be isolated from a native source, or from, a recombinant source where the genetically modified host cell is modified to express the nucleic acid sequence encoding the protein. Alternatively, the Cas protein can be produced using cell free protein expression systems, or be synthetically produced. Effector Cas nucleases may be isolated and introduced into a heterologous cell, or may be modified from its native form to exhibit a different type or magnitude of activity than what it would exhibit in its native source. Such modifications include but are not limited to: fragments, variants, substitutions, deletions, and insertions.

Fragments and variants of Cas endonucleases and Cas effector proteins can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

The Cas endonuclease can comprise a modified form of the Cas polypeptide. The modified form of the Cas polypeptide can include an amino acid change (e.g., deletion, insertion, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797 published 6 Mar. 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas effector protein can be fused to a heterologous sequence to induce or modify activity.

A catalytically active or inactive Cas protein, such as the Cas-alpha protein described herein, can also be in fusion with a molecule that directs editing of single or multiple bases in a polynucleotide sequence, for example a site-specific deaminase that can change the identity of a nucleotide, for example from C·G to T·A or an A·T to G·C (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. A base editing fusion protein may comprise, for example, an active (double strand break creating), partially active (nickase) or deactivated (catalytically inactive) Cas-alpha endonuclease and a deaminase (such as, but not limited to, a cytidine deaminase, an adenine deaminase, APOBEC1, APOBEC3A, BE2, BE3, BE4, ABEs, or the like). Base edit repair inhibitors and glycosylase inhibitors (e.g., uracil glycosylase inhibitor (to prevent uracil removal)) are contemplated as other components of a base editing system, in some embodiments.

### Guide Polynucleotides

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide may be engineered or synthetic.

The guide polynucleotide includes a chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The guide polynucleotide can be a double molecule (also referred to as duplex guide polynucleotide) comprising a crNucleotide sequence (such as a crRNA) and a tracrNucleotide (such as a tracrRNA) sequence. In some cases, there is a linker polynucleotide that connects the crRNA and tracrRNA to form a single guide, for example an sgRNA.

### Gene Editing

The process for editing a genomic sequence combining DSB and modification templates generally comprises: introducing into a host cell a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas-gRNA complexes, has been described, for example in US20150082478 published on 19 Mar. 2015, WO2015026886 published on 26 Feb. 2015, WO2016007347 published 14 Jan. 2016, and WO/2016/025131 published on 18 Feb. 2016.

Some uses for guide RNA/Cas endonuclease systems have been described (see for example: US20150082478 A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

Described herein are methods for genome editing with a Cas endonuclease and complexes with a Cas endonuclease and a guide polynucleotide. Following characterization of the guide RNA and PAM sequence, components of the endonuclease and associated CRISPR RNA (crRNA) may be utilized to modify chromosomal DNA in other organisms including plants. To facilitate optimal expression and nuclear localization (for eukaryotic cells), the genes comprising the complex may be optimized as described in WO2016186953 published 24 Nov. 2016, and then delivered into cells as DNA expression cassettes by methods known in the art. The components necessary to comprise an active complex may also be delivered as RNA with or without modifications that protect the RNA from degradation or as mRNA capped or uncapped (Zhang, Y. et al., 2016, Nat. Commun. 7:12617) or Cas protein guide polynucleotide complexes (WO2017070032 published 27 Apr. 2017), or any combination thereof. Additionally, a part or part(s) of the complex and crRNA may be expressed from a DNA construct while other components are delivered as RNA with or without modifications that protect the RNA from degradation or as mRNA capped or uncapped (Zhang et al. 2016 Nat. Commun. 7:12617) or Cas protein guide polynucleotide complexes (WO2017070032 published 27 Apr. 2017) or any combination thereof. To produce crRNAs in-vivo, tRNA derived elements may also be used to recruit endogenous RNAses to cleave crRNA transcripts into mature forms capable of guiding the complex to its DNA target site, as described, for example, in WO2017105991 published 22 Jun. 2017. Nickase complexes may be utilized separately or concertedly to generate a single or multiple DNA nicks on one or both DNA strands. Furthermore, the cleavage activity of the Cas endonuclease may be deactivated by altering key catalytic residues in its cleavage domain (Sinkunas, T. et al., 2013, EMBO J. 32:385-394) resulting in a RNA guided helicase that may be used to enhance homology directed repair, induce transcriptional activation, or remodel local DNA structures. Moreover, the activity of the Cas cleavage and helicase domains may both be knocked-out and used in combination with other DNA cutting, DNA nicking, DNA binding, transcriptional activation, transcriptional repression, DNA remodeling, DNA deamination, DNA unwinding, DNA recombination enhancing, DNA integration, DNA inversion, and DNA repair agents.

The transcriptional direction of the tracrRNA for the CRISPR-Cas system (if present) and other components of the CRISPR-Cas system (such as variable targeting domain, crRNA repeat, loop, anti-repeat) can be deduced as described in WO2016186946 published 24 Nov. 2016, and WO2016186953 published 24 Nov. 2016.

As described herein, once the appropriate guide RNA requirement is established, the PAM preferences for each new system disclosed herein may be examined. If the cleavage complex results in degradation of the randomized PAM library, the complex can be converted into a nickase by disabling the ATPase dependent helicase activity either through mutagenesis of critical residues or by assembling the reaction in the absence of ATP as described previously (Sinkunas, T. et al, 2013, EMBO J. 32:385-394). Two regions of PAM randomization separated by two protospacer targets may be utilized to generate a double-stranded DNA break which may be captured and sequenced to examine the PAM sequences that support cleavage by the respective complex.

In one embodiment, the invention describes a method for modifying a target site in the genome of a cell, the method comprising introducing into a cell at least one PGEN described herein, and identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, the chemical alteration of at least one nucleotide, and (v) any combination of (i)-(iv).

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10:957-963).

One or more targeted edits can be introduced in the genome through CRISPR-Cas, or another targeted site-directed mutagenesis approach, by modifying the genomic locus or loci having a heterologous polynucleotide comprising a sequence at least 90% identical to a polynucleotide selected from the group consisting of: SEQ ID NOs: 1-287, or a heterologous polypeptide comprising a sequence at least 90% identical to a polypeptide selected from the group consisting of: SEQ ID NOs. 288-567.

Introduction of System Components into a Cell

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, bacterial-mediated transformation (e.g., Agrobacterium-mediated transformation Ochrobacter-mediated transformation, Rhizobiales-mediated transformation), direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

Delivery of a polynucleotide into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome without using a screenable marker phenotype. Such methods can be viewed as directly analyzing a target sequence to detect any change in the target sequence, including but not limited to PCR methods, sequencing methods, nuclease digestion, Southern blots, and any combination thereof.

### Yield and Agronomic Traits

### Drought

In some cases, a plant resulting from seeds or other plant elements transformed with a transgene can exhibit a physiological change, such as a compensation of the stress-induced reduction in photosynthetic activity. Fv/Fm tests whether or not plant stress affects photosystem II in a dark adapted state. Fv/Fm is one of the most commonly used chlorophyll fluorescence measuring parameter. The Fv/Fm test is designed to allow the maximum amount of the light energy to take the fluorescence pathway. It compares the dark-adapted leaf pre-photosynthetic fluorescent state, called minimum fluorescence, or Fo, to maximum fluorescence called Fm. In maximum fluorescence, the maximum number of reaction centers have been reduced or closed by a saturating light source. In general, the greater the plant stress, the fewer open reaction centers available, and the Fv/Fm ratio is lowered. Fv/Fm is a measuring protocol that works for many types of plant stress. For example, there would be a difference in the Fv/Fm after exposure of a transgene treated plant that had been subjected to heat shock or drought conditions, as compared to a corresponding control, a genetically identical plant that does not contain the transgenes grown in the same conditions. In some cases, the transgene-associated plant as disclosed herein can exhibit an increased change in photosynthetic activity AFv(AFv/Fm) after heat-shock or drought stress treatment, for example 1, 2, 3, 4, 5, 6, 7 days or more after the heat-shock or drought stress treatment, or until photosynthesis ceases, as compared with corresponding control plant of similar developmental stage but not comprising the transgene. For example, a plant having a transgene able to confer heat and/or drought-tolerance can exhibit a AFv/Fm of from about 0.1 to about 0.8 after exposure to heat-shock or drought stress or a AFv/Fm range of from about 0.03 to about 0.8 under one day, or 1, 2, 3, 4, 5, 6, 7, or over 7 days post heat-shock or drought stress treatment, or until photosynthesis ceases. In some embodiments, stress-induced reductions in photosynthetic activity can be compensated by at least about 0.25% (for example, at least about 0.5%>, between 0.5%> and 1%>, at least about 1%), between 1%> and 2%, at least about 2%, between 2% and 3%, at least about 3%, between 3% and 5%, at least about 5%, between 5% and 10%, at least about 8%, at least about 10%), between 10% and 15%, at least about 15%, between 15% and 20%, at least about 20%, between 20$ and 25%, at least about 25%, between 25% and 30%, at least about 30%, between 30% and 40%, at least about 40%, between 40% and 50%, at least about 50%, between 50% and 60%, at least about 60%, between 60% and 75%, at least about 75%, between 75% and 80%, at least about 80%, between 80% and 85%, at least about 85%, between 85% and 90%, at least about 90%, between 90% and 95%, at least about 95%, between 95% and 99%, at least about 99% or at least 100%) as compared to the photosynthetic activity decrease in a corresponding reference agricultural plant following heat shock conditions. Significance of the difference between transgene-associated and reference agricultural plants can be established upon demonstrating statistical significance, for example at p<0.05 with an appropriate parametric or non-parametric statistic, e.g., Chi-square test, Student's t-test, Mann-Whitney test, or F-test based on the assumption or known facts that the transgene-associated plant and reference agricultural plant have identical or near identical genomes (isoline comparison).

Increased heat and/or drought tolerance can be assessed with physiological parameters including, but not limited to, increased height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, wilt recovery, turgor pressure, or any combination thereof, as compared to a reference agricultural plant grown under similar conditions.

### Nutrient Limitation

Transgenes described herein may also confer to the plant an increased ability to grow in nutrient limiting conditions, for example by solubilizing or otherwise making available to the plants macronutrients or micronutrients that are complexed, insoluble, or otherwise in an unavailable form. Crop plant nutrients include, without limitation, nitrogen, phosphorous, and potassium.

In one embodiment, a plant is transformed with a transgene that confers increased ability to liberate and/or otherwise provide to the plant with nutrients selected from the group consisting of phosphate, nitrogen, potassium, iron, manganese, calcium, molybdenum, vitamins, or other micronutrients. Such a plant can exhibit increased growth in soil comprising limiting amounts of such nutrients when compared with reference agricultural plant. Differences between the transgene-associated plant and reference agricultural plant can be measured by comparing the biomass of the two plant types grown under limiting conditions, or by measuring the physical parameters described above. Therefore, in one embodiment, the plant comprising a transgene shows increased tolerance to nutrient limiting conditions as compared to a reference agricultural plant grown under the same nutrient limited concentration in the soil, as measured for example by increased biomass or seed yield of at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%), between 200% and 300%, at least 300% or more, when compared with isoline plants grown under the same conditions.

In another embodiment, the plant containing the transgene is able to grown under nutrient stress conditions while exhibiting no difference in the physiological parameter compared to a plant that is grown without nutrient stress. In some embodiments, such a plant will exhibit no difference in the physiological parameter when grown with 2-5% less nitrogen than average cultivation practices on normal agricultural land, for example, at least 5-10% less nitrogen, at least 10-15% less nitrogen, at least 15-20% less nitrogen, at least 20-25% less nitrogen, at least 25-30% less nitrogen, at least 30-35% less nitrogen, at least 35-40% less nitrogen, at least 40-45% less nitrogen, at least 45-50% less nitrogen, at least 50-55% less nitrogen, at least 55-60% less nitrogen, at least 60-65% less nitrogen, at least 65-70% less nitrogen, at least 10-15% less nitrogen, at least 80-85% less nitrogen, at least 85-90% less nitrogen, at least 90-95% less nitrogen, or less, when compared with crop plants grown under normal conditions during an average growing season.

### Water Use Efficiency

The host plants transformed with the transgene may also show improvements in their ability to utilize water more efficiently. Water use efficiency is a parameter often correlated with drought tolerance. Water use efficiency (WUE) is a parameter often correlated with drought tolerance, and is the CO2 assimilation rate per amount of water transpired by the plant. An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a low water potential, are more efficient and thereby are able to produce more biomass and economic yield in many agricultural systems. An increased water use efficiency of the plant relates in some cases to an increased fruit/kernel size or number.

Therefore, in one embodiment, the plants described herein exhibit an increased water use efficiency (WUE) when compared with a reference agricultural plant grown under the same conditions. For example, the transgene may provide an increase in WUE to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions. Such an increase in WUE can occur under conditions without water deficit, or under conditions of water deficit, for example, when the soil water content is less than or equal to 60% of water saturated soil, for example, less than or equal to 50%, less than or equal to 40%, less than or equal to 30%, less than or equal to 20%, less than or equal to 10% of water saturated soil on a weight basis. In some embodiments, the plants inoculated with the endophytic population show increased yield under non-irrigated conditions, as compared to reference agricultural plants grown under the same conditions.

In a related embodiment, the plant comprising the transgene can have a higher relative water content (RWC), than a reference agricultural plant grown under the same conditions.

### Plant Breeding

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization.

The present disclosure finds use in the breeding of plants comprising one or more introduced traits, or edited genomes.

A non-limiting example of how two traits can be stacked into the genome at a genetic distance of, for example, 5 cM from each other is described as follows: A first plant comprising a first transgenic target site integrated into a first DSB target site within the genomic window and not having the first genomic locus of interest is crossed to a second transgenic plant, comprising a genomic locus of interest at a different genomic insertion site within the genomic window and the second plant does not comprise the first transgenic target site. About 5% of the plant progeny from this cross will have both the first transgenic target site integrated into a first DSB target site and the first genomic locus of interest integrated at different genomic insertion sites within the genomic window. Progeny plants having both sites in the defined genomic window can be further crossed with a third transgenic plant comprising a second transgenic target site integrated into a second DSB target site and/or a second genomic locus of interest within the defined genomic window and lacking the first transgenic target site and the first genomic locus of interest. Progeny are then selected having the first transgenic target site, the first genomic locus of interest and the second genomic locus of interest integrated at different genomic insertion sites within the genomic window. Such methods can be used to produce a transgenic plant comprising a complex trait locus having at least 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31 or more transgenic target sites integrated into DSB target sites and/or genomic loci of interest integrated at different sites within the genomic window. In such a manner, various complex trait loci can be generated.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and in the specification rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific embodiments of some aspects of the invention. The examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

The following publications are key representatives of the methods involved (Habben et al. 2014; Shi et al. 2017; Wu et al. 2019). Other methods for gene candidate pre-screening and selections, greenhouse testing, managed stress field testing, and other methods, are known in the art.

Rapid advances in transposon tagging, activation tagging, positional cloning, gene and genomic sequencing, burgeoning published literature, model crop species, and the development of managed stress environments, greatly expanded the concepts to test. Together with improved transformation technology, well-characterized promoters and other genetic elements, and efficient vector construction, transgenic maize plants were produced regularly, at reasonable cost, in quantity and diversity enough to form a pipeline for field testing. Genes identified for testing were sourced, for example, from bioinformatics analyses of crop genomes, from new lead discovery, optimization of existing leads, and redesigned gene edits.

### Example 1: Identification of Gene Leads

Attributes of the ideal lead were classified into roughly right biology, right genetics, and right path. Right biology was a large effect size, few to no serious negative secondary traits, and distinctiveness from existing breeding germplasm. Right genetics was dominance, penetrance, stable expressivity, and sometimes event chromosomal location favorable for trait germplasm integration. Right path was simplicity (esp. single gene), technical feasibility, freedom to operate, expected regulatory approval, and frequently also cis-genic (e.g. genes from maize in maize).

Identification and testing of potential gene leads comprised multiple activities: sourcing gene candidates and hypotheses, primary discovery field tests, retesting to validate leads, and then lead expansions, optimizations, and characterization. Primary tests and lead expansions involved the first instances a specific unique gene was field tested, usually with one promoter. Primary testing included both the first year and second year retest confirmation of that gene. Lead expansions, as an extended special category of newly tested genes, involved testing other new genes related to the founding primary test lead, such as gene family members or pathway-network related genes. Optimizations involved testing the same gene as the original lead, but usually in varied DNA cassette configurations and in other germplasm. In practice these three areas of activities overlapped, results of each informing the others. For instance, newly established leads could have been in continued field testing while molecular and physiology mode of action studies, homolog and pathway lead expansions, and construct optimizations were initiated. The annual construct testing profile of primary tests, lead expansions and optimizations showed primary testing (66.8% all constructs), lead expansions (5.5%), and optimizations (27.6%).

Genes selected for testing were placed into constructs for transformation into maize cells, via a method known in the art.

There were approximately 3863 total field tests on approximately 3331 constructs (via 5-10 transformed events per construct), with about 10% of constructs tested more than once, usually as retests in adjacent years. The 3331 unique field-tested constructs here exclude 9.3% of field tested constructs that were: (a) vector backbone or promoter controls; (b) deviations from expected construct designs, such as incomplete ORFs; (c) uncertain gene sources; (d) involving third party confidential technology, and (e) flowering-maturity or stature related. Unique vectors (3331) exceeded 2:1 unique genes (1671) because some genes were used in multiple vectors, and multi-gene molecular stacks typically involved genes already present in previous vectors.

### Example 2: Particle Bombardment Transformation of Corn Plants

In this example, transformation of immature maize embryos via particle bombardment is described. It is understood that a similar protocol may be used for the transformation of other plants, such as (but not limited to): soybean, cotton, canola, wheat, rice, sorghum, or sunflower.

Prior to bombardment, 10-12 DAP immature embryos are isolated from ears of a corn plant and placed on culture medium plus 16% sucrose for three hours to plasmolyze the scutellar cells.

Single plasmids or multiple plasmids may be used for each particle bombardment. In one example, a plurality of plasmids include: 1) a plasmid comprising a donor cassette, 2) a plasmid comprising an expression cassette for the double-strand-break-inducing agent, and 3) a plasmid comprising an expression cassette for the morphogenic factor.

To attach the DNA to 0.6 μm gold particles, the plasmids are mixed by adding 10 μl of each plasmid together in a low-binding microfuge tube (Sorenson Bioscience 39640T). To this suspension, 50 μl of 0.6 μm gold particles (30 μg/μl) and 1.0 μl of Transit 20/20 (Cat No MIR5404, Minis Bio LLC) are added, and the suspension is placed on a rotary shaker for 10 minutes. The suspension is centrifuged at 10,000 RPM (˜9400×g) and the supernatant is discarded. The gold particles are re-suspended in 120 μl of 100% ethanol, briefly sonicated at low power and 10 μl is pipetted onto each carrier disc. The carrier discs are then air-dried to remove all the remaining ethanol. Particle bombardment is performed using a Biolistics PDF-1000, at 28 inches of Mercury using a 425 PSI rupture disc.

After particle bombardment, the immature embryos are selected on 506J medium modified to contain 12.5 g/l mannose and 5 g/l maltose and no sucrose. After 10-12 weeks on selection, plantlets are regenerated and analyzed using qPCR.

Components may be delivered as DNA, RNA, protein, or any combination of the preceding. In one example, Cas9 and the guide RNA were delivered as a ribonucleoprotein (RNP) complex. Previously, we have described a method for Cas9-gRNA delivery in the form of ribonucleoproteins (RNPs) using gold microparticles. We also described a method for activation of broken pre-integrated selectable marker gene through non-homologous end joining (NHEJ) mechanism upon Cas9-gRNA delivery as DNA vectors or RNP complex (WO 2017/070029 A1).

### Example 3: Agrobacterium-Mediated Transformation of Corn Plants

In this example, transformation of immature maize embryos via Agrobacterium mediation is described. It is understood that a similar protocol may be used for the transformation of other plants, such as (but not limited to): soybean, cotton, canola, wheat, rice, sorghum, or sunflower.

### Preparation of Agrobacterium Master Plate.

Agrobacterium tumefaciens harboring a binary donor vector is streaked out from a −80° C. frozen aliquot onto solid 12V medium and cultured at 28° C. in the dark for 2-3 days to make a master plate.

### Growing Agrobacterium on Solid Medium.

A single colony or multiple colonies of Agrobacterium are picked from the master plate and streaked onto a second plate containing 8101 medium and incubated at 28° C. in the dark overnight.

Agrobacterium infection medium (700 medium A; 5 ml) is added to a 14 mL conical tube in a hood. About 3 full loops of Agrobacterium from the second plate are suspended in the tube and the tube was then vortexed to make an even suspension. One mL is transferred to a spectrophotometer tube and the optical density (550 nm) of the suspension is adjusted to a reading of about 0.35-1.0. The Agrobacterium concentration is approximately 0.5 to 2.0×10⁹ cfu/mL. The final Agrobacterium suspension is aliquoted into 2 mL microcentrifuge tubes, each containing about 1 mL of the suspension. The suspensions are then used as soon as possible.

### Growing Agrobacterium on Liquid Medium.

Alternatively, Agrobacterium can be prepared for transformation by growing in liquid medium. One day before infection, a 125 ml flask is prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL). A half loopful of Agrobacterium from a second plate is suspended into the flasks and placed on an orbital shaker set at 200 rpm and incubated at the 28° C. overnight. The Agrobacterium culture is centrifuged at 5000 rpm for 10 min. The supernatant is removed and the Agrobacterium infection medium was added. The bacteria are resuspended by vortex and the optical density (550 nm) of Agrobacterium suspension is adjusted to a reading of about 0.35 to 2.0.

### Maize Transformation

Ears of a maize (Zea mays L.) cultivar are surface-sterilized for 15-20 min in 20% (v/v) bleach (5.25% sodium hypochlorite) plus 1 drop of Tween 20 followed by 3 washes in sterile water. Immature embryos (IEs) are isolated from ears and are placed in 2 ml of the Agrobacterium infection medium. The optimal size of the embryos varies based on the inbred, but for transformation with WUS2 and ODP2 a wide size range of immature embryo sizes could be used. The solution is drawn off and 1 ml of Agrobacterium suspension was added to the embryos and the tube vortexed for 5-10 sec. The microfuge tube is allowed to stand for 5 min in the hood. The suspension of Agrobacterium and embryos are poured onto 7101 (or 562V) co-cultivation medium (see Table 2). Any embryos left in the tube are transferred to the plate using a sterile spatula. The Agrobacterium suspension is drawn off and the embryos placed axis side down on the media. The plate is sealed with Parafilm M® film (moisture resistant flexible plastic, available at Bemis Company, Inc., 1 Neenah Center 4^{th} floor, PO Box 669, Neenah, Wis. 54957) and incubated in the dark at 21° C. for 1-3 days of co-cultivation.

Embryos are transferred to resting medium (605T medium) without selection. Three to 7 days later, they are transferred to maturation medium (289Q medium) supplemented with a selective agent

### Example 4: Morphogenic Factors Improve Transformation Frequency

Morphogenic factor genes (also referred to as “developmental genes”, for example but not limited to: ODP2 and WUS) are desired components of the transformation process: their delivery into plant cells facilitates cell division and significantly increases transformation frequencies. Moreover, these genes allow successful transformation of many elite genotypes, which transformation, otherwise, cannot be effectively accomplished.

Parameters of the transformation protocol can be modified to ensure that the BBM activity is transient. One such method involves precipitating the BBM-containing plasmid in a manner that allows for transcription and expression, but precludes subsequent release of the DNA, for example, by using the chemical PEI.

In one example, the BBM plasmid is precipitated onto gold particles with PEI, while the transgenic expression cassette (UBI::moPAT˜GFPm::PinII; moPAT is the maize optimized PAT gene) to be integrated is precipitated onto gold particles using the standard calcium chloride method.

Briefly, gold particles were coated with PEI as follows. First, the gold particles were washed. Thirty-five mg of gold particles, 1.0 in average diameter (A.S.I. #162-0010), were weighed out in a microcentrifuge tube, and 1.2 ml absolute EtOH was added and vortexed for one minute. The tube was incubated for 15 minutes at room temperature and then centrifuged at high speed using a microfuge for 15 minutes at 4° C. The supernatant was discarded and a fresh 1.2 ml aliquot of ethanol (EtOH) was added, vortexed for one minute, centrifuged for one minute, and the supernatant again discarded (this is repeated twice). A fresh 1.2 ml aliquot of EtOH was added, and this suspension (gold particles in EtOH) was stored at −20° C. for weeks. To coat particles with polyethylamine (PEI; Sigma #P3143), 250 μl of the washed gold particle/EtOH mix was centrifuged and the EtOH discarded. The particles were washed once in 100 μl ddH2O to remove residual ethanol, 250 μl of 0.25 mM PEI was added, followed by a pulse-sonication to suspend the particles and then the tube was plunged into a dry ice/EtOH bath to flash-freeze the suspension, which was then lyophilized overnight. At this point, dry, coated particles could be stored at −80° C. for at least 3 weeks. Before use, the particles were rinsed 3 times with 250 μl aliquots of 2.5 mM HEPES buffer, pH 7.1, with 1× pulse-sonication, and then a quick vortex before each centrifugation. The particles were then suspended in a final volume of 250 μl HEPES buffer. A 25 μl aliquot of the particles was added to fresh tubes before attaching DNA. To attach uncoated DNA, the particles were pulse-sonicated, then 1 μg of DNA (in 5 μl water) was added, followed by mixing by pipetting up and down a few times with a Pipetteman and incubated for 10 minutes. The particles were spun briefly (i.e. 10 seconds), the supernatant removed, and 60 μl EtOH added. The particles with PEI-precipitated DNA-1 were washed twice in 60 μl of EtOH. The particles were centrifuged, the supernatant discarded, and the particles were resuspended in 45 μl water. To attach the second DNA (DNA-2), precipitation using a water-soluble cationic lipid transfection reagent was used. The 45 μl of particles/DNA-1 suspension was briefly sonicated, and then 5 μl of 100 ng/μl of DNA-2 and 2.5 μl of the water-soluble cationic lipid transfection reagent were added. The solution was placed on a rotary shaker for 10 minutes, centrifuged at 10,000 g for 1 minute. The supernatant was removed, and the particles resuspended in 60 μl of EtOH. The solution was spotted onto macrocarriers and the gold particles onto which DNA-1 and DNA-2 had been sequentially attached were delivered into scutellar cells of 10 DAP Hi-II immature embryos using a standard protocol for the PDS-1000. For this experiment, the DNA-1 plasmid contained a UBI::RFP::pinII expression cassette, and DNA-2 contained a UBI::CFP::pinII expression cassette. Two days after bombardment, transient expression of both the CFP and RFP fluorescent markers was observed as numerous red & blue cells on the surface of the immature embryo. The embryos were then placed on non-selective culture medium and allowed to grow for 3 weeks before scoring for stable colonies. After this 3-week period, 10 multicellular, stably-expressing blue colonies were observed, in comparison to only one red colony. This demonstrated that PEI-precipitation could be used to effectively introduce DNA for transient expression while dramatically reducing integration of the PEI-introduced DNA and thus reducing the recovery of RFP-expressing transgenic events. In this manner, PEI-precipitation can be used to deliver transient expression of BBM and/or WUS2.

For example, the particles are first coated with UBI::BBM::pinII using PEI, then coated with UBI::moPAT˜YFP using a water-soluble cationic lipid transfection reagent, and then bombarded into scutellar cells on the surface of immature embryos. PEI-mediated precipitation results in a high frequency of transiently expressing cells on the surface of the immature embryo and extremely low frequencies of recovery of stable transformants Thus, it is expected that the PEI-precipitated BBM cassette expresses transiently and stimulates a burst of embryogenic growth on the bombarded surface of the tissue (i.e. the scutellar surface), but this plasmid will not integrate. The PAT˜GFP plasmid released from the Ca++/gold particles is expected to integrate and express the selectable marker at a frequency that results in substantially improved recovery of transgenic events. As a control treatment, PEI-precipitated particles containing a UBI::GUS::pinII (instead of BBM) are mixed with the PAT˜GFP/Ca++ particles. Immature embryos from both treatments are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

As an alternative method, the BBM plasmid is precipitated onto gold particles with PEI, and then introduced into scutellar cells on the surface of immature embryos, and subsequent transient expression of the BBM gene elicits a rapid proliferation of embryogenic growth. During this period of induced growth, the explants are treated with Agrobacterium using standard methods for maize (see Example 1), with T-DNA delivery into the cell introducing a transgenic expression cassette such as UBI::moPAT˜GFPm::pinII. After co-cultivation, explants are allowed to recover on normal culture medium, and then are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

It may be desirable to “kick start” callus growth by transiently expressing the BBM and/or WUS2 polynucleotide products. This can be done by delivering BBM and WUS2 5′-capped polyadenylated RNA, expression cassettes containing BBM and WUS2 DNA, or BBM and/or WUS2 proteins. All of these molecules can be delivered using a biolistics particle gun. For example 5′-capped polyadenylated BBM and/or WUS2 RNA can easily be made in vitro using Ambion's mMessage mMachine kit. RNA is co-delivered along with DNA containing a polynucleotide of interest and a marker used for selection/screening such as Ubi::moPAT˜GFPm::PinII. It is expected that the cells receiving the RNA will immediately begin dividing more rapidly and a large portion of these will have integrated the agronomic gene. These events can further be validated as being transgenic clonal colonies because they will also express the PAT˜GFP fusion protein (and thus will display green fluorescence under appropriate illumination). Plants regenerated from these embryos can then be screened for the presence of the polynucleotide of interest.

Use of morphogenic factors, e.g., BBM and/or WUS, had several advantages in our experiments. First, some genotypes are recalcitrant to some types of transformation. Second, forcing cells into division increases the chances of successful outcome, as it activates DSB repair mechanisms. Third, chromosomes positioning in the nucleus is not random but occupy specific territories specific to both cell type and tissue type. Moreover, chromatin organization and interaction are different and depend on specific cell cycle stages. For example, G1 stage is transcriptionally active and characterized by a short range intrachromosomal contact compare to long-range contacts. By association of Hi-C data with cell cycle progression, the prevalence of local contacts during interphase and the enrichment of long-range mitotic contacts during mitosis and early G1 stages was affirmed. Therefore, expression of morphogenic genes, which pushes cell to replication (S-phase) and to cell division likely increases chances of “improper” DSB repair and generation of the desired chromosomal rearrangements.

Other cell cycle proteins and transcription factors may also contribute to the successful outcome.

### Example 5: Gene Expression Strategies

Gene expression strategy is an integral component of gene testing hypotheses. The desired expression pattern was however guided by the gene discovery source, such as activation tagging or specific literature reports. Most primary test gene concepts involved a single gene overexpression (88.2%), of these often using the well-known ubiquitin promoter UB1ZM PRO (65.4%), or moderate OE promoters such as ZM-GOS2 PRO (4.3%), or OS-ACTIN PRO (0.6%). These moderate constitutive promoters were often deliberately used with regulatory genes, especially transcription factors, to mitigate perceived risk of negative effects sometimes observed by excessive expression. The remaining single gene OE primary tests were spread across dozens of promoters or variants. Some single gene primary test concepts (2.5%) required more targeted tissue-preferred or developmental-temporal expression patterns, usually involving root, vegetative-leaf, stalk or flowering preferences, drought or nitrogen inducibility, or diurnal temporal patterns. Such targeted expression was however more commonly employed for lead optimizations. In 11.8% of primary tests the goal was rather to reduce or eliminate expression. The most common approach was to drive RNAi, using the UB1ZM PRO (71%) or other constitutive promoters, although a few were targeted promoters. Gene editing enabled many gene-reducing strategies, such as whole gene knockouts, frameshifts, and promoter deletions.

Multi-gene constructs stacks were 8.7% of all tests, which used different promoters in the stack to avoid vector or transformant instability, and sometimes mixed expression strategies. Genetics stacks were 2.4%, usually containing multiple validated leads. Gene editing was 0.4%, using native or emplaced promoters. Most constructs were greenhouse sampled before field testing for qRT-PCR analysis to verify transgene expression, but for most RNAi experiments, the endogenous gene expression reduction was quantified afterwards for only the positive field-performing constructs.

An estimated 1671 unique genes were field tested. The total number with same gene variants increases approximately 10% when considering alleles, altered peptides, genomic regions, truncations, fusions, and RNAi sub-fragments. Most genes involved complete CDS ORFs, many being codon-optimized for maize expression (designated by “MO”). A total of 47 species contributed genes. Plant genes comprised 97.2%, with 59.5% monocot and 37.5% dicot. Bacteria comprised 1.5% and fungi 1.2%. Of plant genes, maize genes were 53.0%, with the next top three species being Arabidopsis (36.6%), rice (6.1%) and sorghum (1.8%). About 860 maize genes were tested, representing just 2.2% of the maize genome at 40K gene estimate. Similarly, the 595 Arabidopsis genes tested are about 2.2% of that 27K gene genome.

### Example 5: Model Systems

The chief model systems were forward genetic transgene activation-tagging in Arabidopsis and Rice, and reverse genetics transgenic (usually overexpression) screening in short-cycling maize, Arabidopsis, and rice. Bacterial reverse-genetics screens were used for specific concept areas thought translatable to plants, such as reactive oxygen species tolerance. Reverse genetics approaches ranged from those involving strong specific hypotheses to less-specific hypotheses and thus more ‘open’ screens, especially when involving RNA profiling, pathway-network affiliations, or homologs and family member information.

### Example 6: Field Testing

All field tests measured yield under a range of environmental conditions across field locations, with water availability often being the chief abiotic stress factor affecting yield, whether due to limited ambient rainfall or purposeful managed drought stress.

Hypothesis concepts represented by these constructs were nominated to field testing in these main target trait categories: DRT or yield under drought or simply abiotic stress (57%); NUE or Nitrogen Use Efficiency (34.7%); YE or Yield Enhancement, including yield stability and dual DRT-NUE testing (7.4%); maturity or flowering (0.4%); plant stature (0.2%); and others, mostly various abiotic stresses such as temperature (0.5%).

Most first time field tests were assembled into annual cohort subsets sharing a single transformation inbred line, a single top-cross tester, and a common nursery source. It was commonly observed that significantly positive performing constructs are outnumbered by significantly negative performing constructs, although in this example the split between all nominally positive or negative performance is roughly even. The magnitude of negative performance is typically greater than the magnitude of positive performance, something also commonly seen.

Constructs meeting performance criteria in more than one location were generally retested to validate in the following year, especially if they did not show negative impacts on secondary traits such as standability, maturity, plant height, or grain moisture. Across all years about 10% of constructs were retested. Events selection for retesting was affected by many other factors including: concept redundancy with other test constructs, field testing space and budget constraints, nursery seed availability, permits and timing, retirement of current vector backbones or transformation germplasm, and perceived regulatory burdens.

Because second year testing was more rigorous, it was usually observed that exceptionally high performing first-year events moderated performance in second year tests, often to below sustaining interest. In contrast, marginally performing first year events rarely to never improved performance. The most enduring prospective leads performed moderately high (e.g., 4-5 bu/ac or more) at multiple locations in the first-year tests and repeated similarly in second or subsequent year tests.

### Example 7: Validated Leads

After two years of positive field test performance, a gene construct test was generally deemed ‘validated’, which meant it was considered worthy of investing further field testing, physiological and mode-of-action characterization, and lead optimization. Historically 1.3% (22/1671) of all genes became validated. Because the BLUP p-value was 0.1 in both the first and second year of testing, the validated leads rate tracks right on the cusp of statistical significance (when roughly calculated as 0.1×0.1=0.01). However, as noted above in practice advancement decisions were more complex than this. Once validated, continued testing in additional years pared down the number of actively pursued validated leads. Lead discontinuation was driven by this additional field testing, with particular added scrutiny for germplasm interactions, secondary traits, and effect size relative to commercial potential, ability or not to be sufficiently performance optimized, and expected deregulation costs. The pipeline uncovered other genes with confirmed biological effects. The overall gene validation rate was 1.3% for primary and lead expansion concepts.

Whether YE, NUE, or DRT, these are all clearly complex interconnected traits, with myriad subtending connections, and the measurement was always yield. Positive yield determinations were selected as: 5-10% increase in yield at normal water-nitrogen management; maintained yield at 30% less nitrogen; and 10% yield increase under moderate-severe (drought) stress. The trait categories YE had a 4% success rate, DRT 1.6% and NUE 0.7%. Many genes that performed under drought were dropped, so the drought or abiotic stress validation rate is well below the success rate for demonstrating some drought tolerance. Validated drought leads generally contain those that best preserved yield parity under normal water environments, a key criterion for successful drought leads. Plant hormones have long been implicated in governing complex plant biology, and many validated leads are hormone related.

### Example 8: Lead Expansion

Lead expansions were efforts to increase gene testing space around specific validated gene leads. Lead expansions involve new untested genes and were really a specialized subset of primary testing. Lead expansion followed several main strategies. Lead expansion commonly involved testing other gene family members from the same species (usually maize, and sometimes allelic variants), or from other organisms, especially closely related plants. There was a balanced strategy to include greater sequence diversity, but not so distant that function likely differed entirely, although where this boundary lay itself became a discovery exercise. Gene expression analysis was also used to priority rank gene family members. Lead expansion often employed gene expression analyses of the original lead-bearing transgenic plants, to seek additional pathway-network genes that may be specifically and directly regulated by the lead OE, possibly enriching for other genes that drive or convey the desired positive effects of the original lead.

### Example 9: Lead Optimization

Lead optimizations aimed to develop validated leads, by increasing their product level performance, mitigating any negative phenotypes, and learning more about the lead's functional stability, germplasm and biotech trait combinability, and capabilities and ideal product placement. Optimization efforts frequently involved new vector designs and transformations. The ZMM28 lead for example was improved by optimization using the ZM-GOS2 promoter and filtering chromosome insertion sites to select superior events. Initially lead optimizations were small-scale redesign efforts, primarily to meet product placement and regulatory objectives. Optimizations grew to comprise large structured experiments, both dominating the pipeline and emphasizing improved performance. Later still gene editing itself became the chief single-step optimization, when it emerged as the preferred product development alternative to transgenics.

While small scale lead optimizations brought successes, a more systematic approach to optimization was. We sought to vary gene expression and chromosome locations through creating a matrix of constructs and new transformations using diverse promoters, regulatory elements, introns, and terminators. Random insertions to vary chromosome locations were used, but subsequent matrix optimization experiments used SSI (site-specific integration).

We claim:

1. A method of improving yield of an elite maize plant, the method comprising:
(a) introducing into an elite maize plant cell a heterologous polynucleotide comprising a sequence at least 95% identical to SEQ ID NO: 1, or the heterologous polynucleotide encoding a heterologous polypeptide comprising a sequence at least 95% identical to SEQ ID NO: 288;
(b) regenerating an elite maize plant therefrom;
(c) subjecting the elite maize plant to an environmental condition; and
(d) assessing one or more characteristics of the elite maize plant;
wherein the one or more characteristics demonstrate increased yield or improved agronomic characteristic of the plant, as compared to an isoline maize plant not comprising said heterologous polynucleotide or heterologous polypeptide.

2. The method of claim 1, wherein the heterologous polynucleotide is operably linked to an expression element.

3. The method of claim 2, wherein the expression element regulates expression of the polynucleotide in a tissue-specific manner.

4. The method of claim 2, wherein the expression element regulates expression of the polynucleotide in a constitutive manner.

5. The method of claim 1, wherein the introducing of step (a) is achieved via particle bombardment or bacterial-mediated transformation and further comprising introducing into the cell a morphogenic factor.

6. The method of claim 5, further comprising introducing into the cell a Cas endonuclease and guide polynucleotide.

7. The method of claim 1, wherein the environmental condition is selected from the group consisting of: water limitation, water overabundance, nitrogen limitation, and nitrogen overabundance.

8. The method of claim 1, wherein the one or more characteristics is selected from the group consisting of: plant height, plant biomass, canopy architecture, root biomass, root length, root architecture, water use efficiency, water uptake efficiency, nitrogen use efficiency, nitrogen uptake, nitrogen metabolism, leaf color, pollen mass, seed weight, number of seeds, moisture content of seeds.

9. The method of claim 1, wherein the plant is maize.

10. A recombinant construct comprising: a polynucleotide comprising a sequence at least 95% identical to SEQ ID NO: 1, operably linked to a heterologous expression element.

11. A method of increasing grain yield and/or one more agronomic characteristics of an elite maize plant, the method comprising introducing one or more nucleotide modifications through a targeted site-directed modification at a genomic locus of an elite maize plant, wherein the genomic locus comprises a polynucleotide encoding a polypeptide that is at least 95% identical to SEQ ID NO: 288, such that its expression and/or activity is modified; and wherein the grain yield and/or agronomic characteristic is improved compared to a control maize plant not comprising the one or more introduced modifications, and wherein the method further comprises introducing into the elite maize plant a morphogenic factor.

12. The method of claim 11, wherein the one or more introduced nucleotide changes at the genomic loci is selected from the group consisting of:
(a) generation of one or more alternative spliced transcripts of a polynucleotide encoding the polypeptide;
(b) deletion of one or more domains of the polypeptide;
(c) frameshift mutation in one or more exons of a polynucleotide encoding the polypeptide; and
(d) deletion of a substantial portion of the polynucleotide encoding the polypeptide; and
(e) modification of one or more nucleotides or deletion of a regulatory element operably linked to the expression of the polynucleotide encoding the polypeptide,
wherein the regulatory element is present within a promoter, intron, 3′UTR, terminator or a combination thereof.

13. The method of claim 11, wherein the expression and/or activity modification is selected from the group consisting of:
(a) reduced expression of a polynucleotide encoding the polypeptide;
(b) reduced activity of the polypeptide; and
(c) repression of an enhancer motif present within a regulatory region encoding the polypeptide.
